(19) United States
(12) Patent Application Publication
(10) Pub. No.: US 2023/0129394 A1
(43) Pub. Date: Apr. 27, 2023

(54) METHODS OF TREATING OR PREVENTING VIRAL INFECTION WITH FLUORINATED AlphaV INTEGRIN ANTAGONISTS

(71) Applicant: OcuTerra Therapeutics, Inc., Boston, MA (US)

(72) Inventor: D. Scott Edwards, Bedford, MA (US)

(21) Appl. No.: 17/982,846

(22) Filed: Nov. 8, 2022

Related U.S. Application Data
(63) Continuation of application No. PCT/US2021/032005, filed on May 12, 2021.
(60) Provisional application No. 63/023,377, filed on May 12, 2020.

(51) Int. Cl. A61K 31/444; A61K 31/506; A61K 31/4709; A61K 31/4353; A61P 11/00; A61P 29/00; A61P 31/14

(52) U.S. Cl. CPC A61K 31/444; A61K 31/506; A61K 31/4709; A61K 31/4353; A61P 11/00; A61P 29/00; A61P 31/14

(57) ABSTRACT

The present application relates to use of fluorinated compounds of Formula (I) in treating or preventing a viral infection or a condition or symptom associated with the viral infection.

### RELATED APPLICATIONS

This application is a continuation of international patent application PCT/US2021/032005, filed May 12, 2021, which claims priority to and the benefit of U.S. Ser. No. 63/023,377, filed on May 12, 2020; the entire contents of each of which are incorporated herein by reference.

### BACKGROUND

A number of viruses use cell surface integrins for either attachment or entry into the cell, including human adenovirus, Kaposi's sarcoma-associated herpes virus, HIV-1, rotavirus, West Nile virus, human papilloma virus, and Ebola virus. A recent study revealed that SARS-Cov-2 infection involves binding of the viral spike protein (S protein) to cell surface integrins through a RGD motif formed by the S protein. It is suggested that the RGD motif plays an important role in promoting rapid human to human transmission.

Integrins are a large family of receptors with 24 identified members organized into four sub-groups: collagen receptors, leukocyte-specific receptors, laminin receptors, and extra-cellular matrix proteins having an RGD recognition sequence (e.g., vitronectin, fibronectin, TGFb-LAP (latency associated peptide), and fibrinogen). The integrins with RGD binding sites include αVβ3, αVβ5, αVβ6, αVβ8, α5β1, and α8β1.

Inhibitors of RGD-binding integrins as anti-viral approaches were studied. Binding and infection by the Foot and Mouth Disease virus was inhibited in cultured cells by either antibodies to the αV subunit or to the 33 subunit. Cellular entry by West Nile virus was blocked by antibodies to αVβ3 and by a soluble form of the receptor. Kaposi's sarcoma-associated herpes virus infection was shown to be inhibited by αVβ3 antibodies as well as small RGD containing peptides. Small molecule inhibitors of RGD-binding integrins are being developed, including cilengitide, MK-0429, risuteganib, SF0166, and THR-687, for various non-viral indications. However, these is a considerable range of potency and selectivity among the available αV integrin inhibitors.

In addition to slowing or preventing viral entry, RGD-binding integrin inhibitors may have favorable impact on the disease process. Impact of integrin inhibition on the development of acute respiratory distress syndrome (ARDS) has been reported. In addition, αVβ3 is a key regulator of macrophage inflammatory response: its activation maintains chronic inflammatory processes, while its inhibition may allow macrophages to escape the inflammatory state. Inhibition of αVβ5 in mice and rats has shown effects in preventing acute lung injury. αVβ6 activates latent TGF-beta in the lungs, and mice lacking this integrin were completely protected from pulmonary edema in a model of bleomycin-induced acute lung injury. Thus, there is a need for inhibitors of RGD-binding integrins for the treatment and prevention of viral infection, such as infection by coronaviruses, and for improving the symptoms caused by or associated with viral infection. The present application addresses the need.

### SUMMARY

The present application provides use of an integrin inhibitor (or integrin antagonist), optionally in combination with a second therapeutic agent, for treating or preventing a viral infection or a condition or symptom associated with the viral infection.

The present application provides a method of treating or preventing a viral infection or a condition or symptom associated with the viral infection in a subject in need thereof, comprising administering to the subject a therapeutically effective amount of an integrin inhibitor (or integrin antagonist), optionally in combination with a second therapeutic agent.

The present application provides use of an integrin inhibitor (or integrin antagonist), optionally in combination with a second therapeutic agent, in the manufacture of a medicament for treating or preventing a viral infection or a condition or symptom associated with the viral infection.

The present application provides an integrin inhibitor (or integrin antagonist), optionally in combination with a second therapeutic agent, for the treatment or prevention of a viral infection or a condition or symptom associated with the viral infection.

The present application provides an integrin inhibitor (or integrin antagonist), optionally in combination with a second therapeutic agent, in the manufacture of a medicament for the treatment or prevention of a viral infection or a condition or symptom associated with the viral infection.

The present application provides use of a compound of Formula (I):

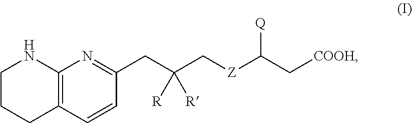

or a pharmaceutically acceptable salt or solvate thereof, for treating or preventing a viral infection or a condition or symptom associated with the viral infection, wherein:

Z is

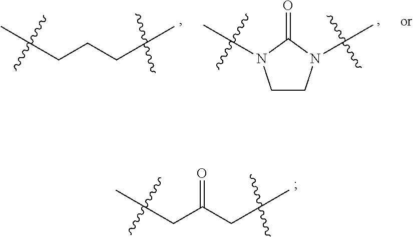 [SMILES: *CCC* ; *N1CCN(*)C1=O ; *CC(=O)C*]

Q is

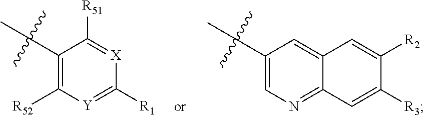

X is CR₄ or N;

Y is CR₄ or N;

R and R′ are each independently H or F, or R and R′, together with the carbon atom to which they are attached, form a 3—or 4-membered carbocyclic or heterocyclic ring;

R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;

R₂ and R₃ are each independently H, F, CH₂F, CHF₂, or CF₃, provided that one of R₂ and R₃ is not H;

each R₄ is independently H, CH₂F, CHF₂, or CF₃; and

R₅₁ and R₅₂ are each independently H, F, or Cl; provided that the compound of Formula (I) contains at least one fluorine atom.

The present application provides use of a pharmaceutical composition comprising a compound of the present application or a pharmaceutically acceptable salt or solvate thereof, and a pharmaceutically acceptable carrier or excipient, for treating or preventing a viral infection or a condition or symptom associated with the viral infection.

The present application provides use of a compound of the present application or a pharmaceutically acceptable salt or solvate thereof, in combination with a second therapeutic agent, for treating or preventing a viral infection or a condition or symptom associated with the viral infection.

The present application provides a method of treating or preventing a viral infection or a condition or symptom associated with the viral infection in a subject in need thereof, comprising administering to the subject a therapeutically effective amount of a compound of the present application or a pharmaceutically acceptable salt or solvate thereof.

The present application provides a method of treating or preventing a viral infection or a condition or symptom associated with the viral infection in a subject in need thereof, comprising administering to the subject a therapeutically effective amount of a pharmaceutical composition comprising a compound of the present application or a pharmaceutically acceptable salt or solvate thereof, and a pharmaceutically acceptable carrier or excipient.

The present application provides a method of treating or preventing a viral infection or a condition or symptom associated with the viral infection in a subject in need thereof, comprising administering to the subject a therapeutically effective amount of a compound of the present application or a pharmaceutically acceptable salt or solvate thereof, in combination with a second therapeutic agent.

The present application provides use of a compound of the present application, or a pharmaceutically acceptable salt or solvate thereof, in the manufacture of a medicament for treating or preventing a viral infection or a condition or symptom associated with the viral infection.

The present application provides use of a pharmaceutical composition comprising a compound of the present application or a pharmaceutically acceptable salt or solvate thereof, and a pharmaceutically acceptable carrier or excipient, in the manufacture of a medicament for treating or preventing a viral infection or a condition or symptom associated with the viral infection.

The present application provides use of a compound of the present application or a pharmaceutically acceptable salt or solvate thereof, in combination with a second therapeutic agent, in the manufacture of a medicament for treating or preventing a viral infection or a condition or symptom associated with the viral infection.

The present application provides a compound of the present application or a pharmaceutically acceptable salt or solvate thereof, for the treatment or prevention of a viral infection or a condition or symptom associated with the viral infection.

The present application provides a pharmaceutical composition comprising a compound of the present application or a pharmaceutically acceptable salt or solvate thereof, and a pharmaceutically acceptable carrier or excipient, for the treatment or prevention of a viral infection or a condition or symptom associated with the viral infection.

The present application provides a compound of the present application or a pharmaceutically acceptable salt or solvate thereof, in combination with a second therapeutic agent, for the treatment or prevention of a viral infection or a condition or symptom associated with the viral infection.

The present application provides a compound of the present application or a pharmaceutically acceptable salt or solvate thereof, for use in the manufacture of a medicament for the treatment or prevention of a viral infection or a condition or symptom associated with the viral infection.

The present application provides a pharmaceutical composition comprising a compound of the present application or a pharmaceutically acceptable salt or solvate thereof, and a pharmaceutically acceptable carrier or excipient, for use in the manufacture of a medicament for the treatment or prevention of a viral infection or a condition or symptom associated with the viral infection.

The present application provides a compound of the present application or a pharmaceutically acceptable salt or solvate thereof, in combination with a second therapeutic agent, for use in the manufacture of a medicament for the treatment or prevention of a viral infection or a condition or symptom associated with the viral infection.

In one aspect, the viral infection is mediated by an integrin, such as an RGD-binding integrin. In one aspect, the condition or symptom is a condition or symptom in which an integrin, such as an RGD-binding integrin, is involved.

Unless otherwise defined, all technical and scientific terms used herein have the same meaning as commonly understood by one of ordinary skill in the art to which this application belongs. In the case of conflict, the present specification, including definitions, will control. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art to the present application. In addition, the materials, methods, and examples are illustrative only and are not intended to be limiting. Other features and advantages of the present application will be apparent from the following detailed description and claims.

### DETAILED DESCRIPTION

The present application relates to integrin antagonists, such as a compound of one of the formulae disclosed herein or a compound described herein, useful in the treatment or prevention of a viral infection (e.g., SARS-CoV-2) or a condition or symptom associated with the viral infection (e.g., COVID-19).

In one aspect, the present application provides use of an integrin inhibitor (or integrin antagonist), optionally in combination with a second therapeutic agent, for treating or preventing a viral infection or a condition or symptom associated with the viral infection.

In one aspect, the present application provides a method of treating or preventing a viral infection or a condition or symptom associated with the viral infection in a subject in need thereof, comprising administering to the subject a therapeutically effective amount of an integrin inhibitor (or integrin antagonist), optionally in combination with a second therapeutic agent.

In one aspect, the present application provides use of an integrin inhibitor (or integrin antagonist), optionally in combination with a second therapeutic agent, in the manufacture of a medicament for treating or preventing a viral infection or a condition or symptom associated with the viral infection.

In one aspect, the present application provides an integrin inhibitor (or integrin antagonist), optionally in combination with a second therapeutic agent, for the treatment or prevention of a viral infection or a condition or symptom associated with the viral infection.

In one aspect, the present application provides an integrin inhibitor (or integrin antagonist), optionally in combination with a second therapeutic agent, in the manufacture of a medicament for the treatment or prevention of a viral infection or a condition or symptom associated with the viral infection.

In one aspect, the present application provides use of a compound of Formula (I):

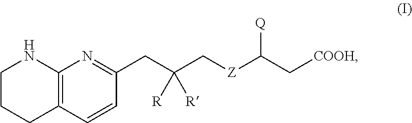

or a pharmaceutically acceptable salt or solvate thereof, for treating or preventing a viral infection or a condition or symptom associated with the viral infection, wherein:

Z is

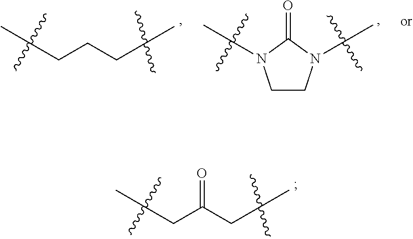 [SMILES: *CCC* ; *N1CCN(*)C1=O ; *CC(=O)C*]

Q is

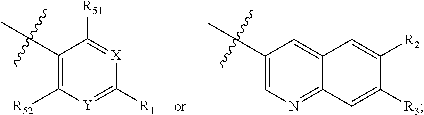

X is CR₄ or N;

Y is CR₄ or N;

R and R′ are each independently H or F, or R and R′, together with the carbon atom to which they are attached, form a 3—or 4-membered carbocyclic or heterocyclic ring;

R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;

R₂ and R₃ are each independently H, F, CH₂F, CHF₂, or CF₃, provided that one of R₂ and R₃ is not H;

each R₄ is independently H, CH₂F, CHF₂, or CF₃; and

R₅₁ and R₅₂ are each independently H, F, or Cl; provided that the compound of Formula (I) contains at least one fluorine atom.

In another aspect, the present application provides use of a pharmaceutical composition comprising a compound of the present application or a pharmaceutically acceptable salt or solvate thereof, and a pharmaceutically acceptable carrier or excipient, for treating or preventing a viral infection or a condition or symptom associated with the viral infection.

In another aspect, the present application provides use of a compound of the present application or a pharmaceutically acceptable salt or solvate thereof, in combination with a second therapeutic agent, for treating or preventing a viral infection or a condition or symptom associated with the viral infection.

In another aspect, the present application provides a method of treating or preventing a viral infection or a condition or symptom associated with the viral infection in a subject in need thereof, comprising administering to the subject a therapeutically effective amount of a compound of the present application or a pharmaceutically acceptable salt or solvate thereof.

In another aspect, the present application provides a method of treating or preventing a viral infection or a condition or symptom associated with the viral infection in a subject in need thereof, comprising administering to the subject a therapeutically effective amount of a pharmaceutical composition comprising a compound of the present application or a pharmaceutically acceptable salt or solvate thereof, and a pharmaceutically acceptable carrier or excipient.

In another aspect, the present application provides a method of treating or preventing a viral infection or a condition or symptom associated with the viral infection in a subject in need thereof, comprising administering to the subject a therapeutically effective amount of a compound of the present application or a pharmaceutically acceptable salt or solvate thereof, in combination with a second therapeutic agent.

In another aspect, the present application provides use of a compound of the present application, or a pharmaceutically acceptable salt or solvate thereof, in the manufacture of a medicament for treating or preventing a viral infection or a condition or symptom associated with the viral infection.

In another aspect, the present application provides use of a pharmaceutical composition comprising a compound of the present application or a pharmaceutically acceptable salt or solvate thereof, and a pharmaceutically acceptable carrier or excipient, in the manufacture of a medicament for treating or preventing a viral infection or a condition or symptom associated with the viral infection.

In another aspect, the present application provides use of a compound of the present application or a pharmaceutically acceptable salt or solvate thereof, in combination with a second therapeutic agent, in the manufacture of a medicament for treating or preventing a viral infection or a condition or symptom associated with the viral infection.

In another aspect, the present application provides a compound of the present application or a pharmaceutically acceptable salt or solvate thereof, for the treatment or prevention of a viral infection or a condition or symptom associated with the viral infection.

In another aspect, the present application provides a pharmaceutical composition comprising a compound of the present application or a pharmaceutically acceptable salt or solvate thereof, and a pharmaceutically acceptable carrier or excipient, for the treatment or prevention of a viral infection or a condition or symptom associated with the viral infection.

In another aspect, the present application provides a compound of the present application or a pharmaceutically acceptable salt or solvate thereof, in combination with a second therapeutic agent, for the treatment or prevention of a viral infection or a condition or symptom associated with the viral infection.

In another aspect, the present application provides a compound of the present application or a pharmaceutically acceptable salt or solvate thereof, for use in the manufacture of a medicament for the treatment or prevention of a viral infection or a condition or symptom associated with the viral infection.

In another aspect, the present application provides a pharmaceutical composition comprising a compound of the present application or a pharmaceutically acceptable salt or solvate thereof, and a pharmaceutically acceptable carrier or excipient, for use in the manufacture of a medicament for the treatment or prevention of a viral infection or a condition or symptom associated with the viral infection.

In another aspect, the present application provides a compound of the present application or a pharmaceutically acceptable salt or solvate thereof, in combination with a second therapeutic agent, for use in the manufacture of a medicament for the treatment or prevention of a viral infection or a condition or symptom associated with the viral infection.

For the use, method, or compound for use of the present application:

In one embodiment, a compound of Formula (I) is of Formula (Ia):

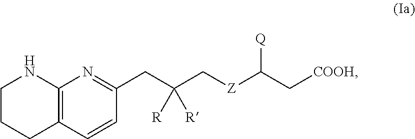

or a pharmaceutically acceptable salt or solvate thereof, wherein:

Z is

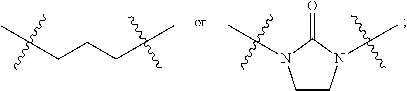 [SMILES: *CCC* ; *N1CCN(*)C1=O]

R and R′ are each independently H or F, or R and R′, together with the carbon atom to which they are attached, form a 3—or 4-membered carbocyclic or heterocyclic ring; Q is

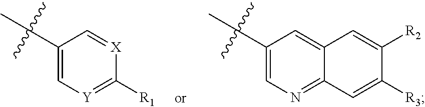

X is CH or N;

Y is CH or N;

R₁ is C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms; and

R₂ and R₃ are each independently H, F, CH₂F, CHF₂, or CF₃, provided that one of R₂ and R₃ is not H,

provided that the compound of Formula (Ia) contains at least one fluorine atom.

For a compound of Formula (Ia):

In one embodiment, Z is

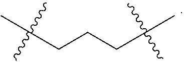 [SMILES: *CCC*]

In another embodiment, Z is

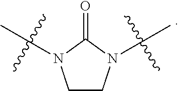 [SMILES: *N1CCN(*)C1=O]

In one embodiment, R and R′ are each H. In another embodiment, R and R′ are each F. In another embodiment, R is H and R′ is F.

In another embodiment, R and R′, together with the carbon atom to which they are attached, form a 3—or 4-membered carbocyclic or heterocyclic ring. In a further embodiment, R and R′, together with the carbon atom to which they are attached, form a 4-membered heterocyclic ring. In a further embodiment, the 4-membered heterocyclic ring is an oxetane ring. For example, the oxetane ring is an oxetan-3-yl ring or oxetan-2-yl ring.

In one embodiment, Q is

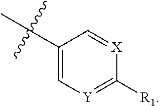

In one embodiment, X is N and Y is CH. In another embodiment, X and Y are each CH. In another embodiment, X and Y are each N.

In one embodiment, R₁ is straight chain C₁-C₄ or branched C₃-C₄ alkyl, and is substituted with 1, 2, 3, 4, 5, 6, 7, 8, or 9 fluorine atoms. In a further embodiment, R₁ is methyl, ethyl, propyl, or butyl, and is substituted with 1, 2, 3, 4, 5, 6, 7, 8, or 9 fluorine atoms. In a further embodiment, R₁ is methyl substituted with 1, 2, or 3 fluorine atoms. In a further embodiment, R₁ is CF₃.

In another embodiment, R₁ is straight chain C₁-C₆ or branched C₃-C₆ alkoxy, and is substituted with 0, 1, 2, 3, 4, 5, 6, or 7 fluorine atoms. In a further embodiment, R₁ is methoxy, ethoxy, propoxy, or butoxy, and is substituted with 0, 1, 2, 3, 4, 5, 6, or 7 fluorine atoms. In a further embodiment, R₁ is methoxy substituted with 0, 1, 2, or 3 fluorine atoms. In a further embodiment, R₁ is OCH₃, OCH₂F, OCHF₂, or OCF₃. In a further embodiment, R₁ is OCHF₂.

In another embodiment, Q is

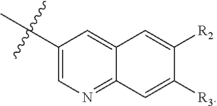

In one embodiment, R₂ is F. In a further embodiment, R₂ is F and R₃ is H. In another embodiment, R₂ is CH₂F, CHF₂, or CF₃.

In one embodiment, R₃ is F. In a further embodiment, R₃ is F and R₂ is H. In another embodiment, R₃ is CH₂F, CHF₂, or CF₃. In a further embodiment, R₃ is CF₃. In a further embodiment, R₃ is CF₃ and R₂ is H.

In one embodiment, R₂ and R₃ are each F.

In one embodiment, Z is

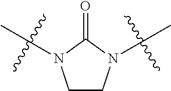 [SMILES: *N1CCN(*)C1=O]

### and Q is

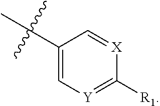

In a further embodiment, Z is

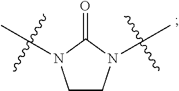 [SMILES: *N1CCN(*)C1=O]

### Q is

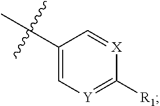

and R and R′ are each H.

In a further embodiment, Z is

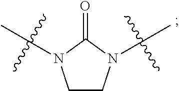 [SMILES: *N1CCN(*)C1=O]

### Q is

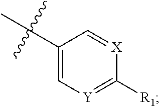

R and R′ are each H; and R₁ is OCH₃, OCH₂F, OCHF₂, or OCF₃. In a further embodiment, X is N and Y is CH; and R₁ is OCHF₂.

In one embodiment, Z is

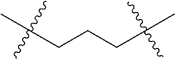 [SMILES: *CCC*]

### and Q is

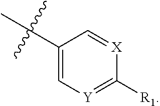

In another further embodiment, Z is

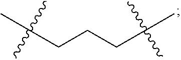 [SMILES: *CCC*]

### Q is

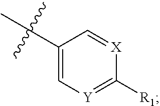

and X and Y are each N. In a further embodiment R₁ is methyl substituted with 1, 2, or 3 fluorine atoms. In a further embodiment R₁ is CF₃.

In a further embodiment, Z is

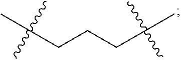 [SMILES: *CCC*]

### Q is

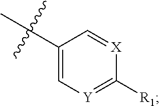

and X is N and Y is CH. In a further embodiment, R₁ is OCH₃, OCH₂F, OCHF₂, or OCF₃. In a further embodiment, R₁ is OCHF₂.

In one embodiment, Z is

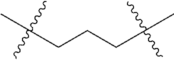 [SMILES: *CCC*]

### and Q is

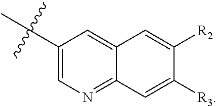

In one embodiment, a compound of Formula (Ia) is of Formula (Ia1):

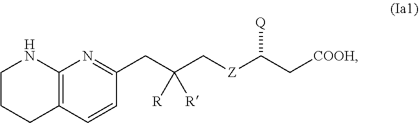

or a pharmaceutically acceptable salt or solvate thereof, wherein each of the variables are as defined above. Compounds of the present application include compounds of Formula (Ia1), wherein the variables are illustrated in the various embodiments of Formula (Ia) above.

In one embodiment, a compound of Formula (I) is of Formula (Ib):

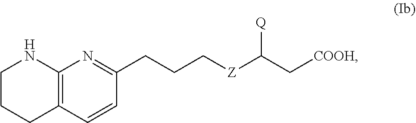

or a pharmaceutically acceptable salt or solvate thereof, wherein:

Z is

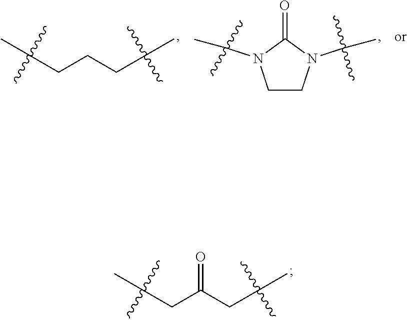 [SMILES: *CCC* ; *N1CCN(*)C1=O ; *CC(=O)C*]

Q is

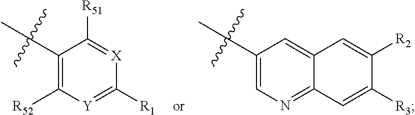

X is CR₄ or N;

Y is CR₄ or N;

R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;

R₂ and R₃ are each independently H, F, CH₂F, CHF₂, or CF₃, provided that one of R₂ and R₃ is not H;

each R₄ is independently H, CH₂F, CHF₂, or CF₃; and

R₅₁ and R₅₂ are each independently H, F, or Cl; provided that the compound of Formula (Ib) contains at least one fluorine atom, and provided that when Z is

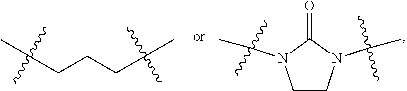 [SMILES: *CCC* ; *N1CCN(*)C1=O]

R₁ is not H, F, or Cl, and R₅₁ and R₅₂ are each H, then at least one of X and Y is CR₄, and R₄ is CH₂F, CHF₂, or CF₃.

For a compound of Formula (Ib):

In one embodiment, Z is

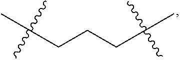 [SMILES: *CCC*]

In another embodiment, Z is

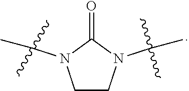 [SMILES: *N1CCN(*)C1=O]

In another embodiment, Z is

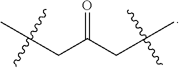 [SMILES: *CC(=O)C*]

In one embodiment, Q is

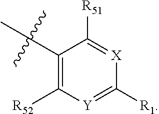

In one embodiment, X is N and Y is CR₄. In another embodiment, X and Y are each CR₄. In another embodiment, X and Y are each N.

In one embodiment, at least one R₄ is H. In one embodiment, at least one R₄ is CH₂F, CHF₂, or CF₃. In a further embodiment, at least one R₄ is CF₃.

In one embodiment, R₁ is H. In another embodiment, R₁ is F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is F or Cl. In another embodiment, R₁ is C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms.

In a further embodiment, R₁ is straight chain C₁-C₄ or branched C₃-C₄ alkyl, and is substituted with 1, 2, 3, 4, 5, 6, 7, 8, or 9 fluorine atoms. In a further embodiment, R₁ is methyl, ethyl, propyl, or butyl, and is substituted with 1, 2, 3, 4, 5, 6, 7, 8, or 9 fluorine atoms. In a further embodiment, R₁ is methyl substituted with 1, 2, or 3 fluorine atoms. In a further embodiment, R₁ is CF₃.

In another further embodiment, R₁ is straight chain C₁-C₆ or branched C₃-C₆ alkoxy, and is substituted with 0, 1, 2, 3, 4, 5, 6, or 7 fluorine atoms. In a further embodiment, R₁ is methoxy, ethoxy, propoxy, or butoxy, and is substituted with 0, 1, 2, 3, 4, 5, 6, or 7 fluorine atoms. In a further embodiment, R₁ is methoxy substituted with 0, 1, 2, or 3 fluorine atoms. In a further embodiment, R₁ is OCH₃, OCH₂F, OCHF₂, or OCF₃. In a further embodiment, R₁ is OCHF₂ or OCF₃.

In one embodiment, R₅₁ and R₅₂ are each H. In another embodiment, one of R₅₁ and R₅₂ is H, and the other is F or Cl. In a further embodiment, one of R₅₁ and R₅₂ is H, and the other is F. In another embodiment, R₅₁ and R₅₂ are each F or Cl.

In another embodiment, Q is

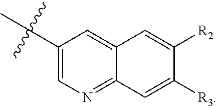

In one embodiment, R₂ is F. In a further embodiment, R₂ is F and R₃ is H. In another embodiment, R₂ is CH₂F, CHF₂, or CF₃.

In one embodiment, R₃ is F. In a further embodiment, R₃ is F and R₂ is H. In another embodiment, R₃ is CH₂F, CHF₂, or CF₃. In a further embodiment, R₃ is CF₃. In a further embodiment, R₃ is CF₃ and R₂ is H.

In one embodiment, R₂ and R₃ are each F.

Any of the substituent groups illustrated above for any of X, Y, Z, Q, R₁, R₂, R₃, R₄, R₅₁, and R₅₂ can be combined with any of the substituent groups illustrated above for the remaining of X, Y, Z, Q, R₁, R₂, R₃, R₄, R₅₁, and R₅₂.

In one embodiment, Q is

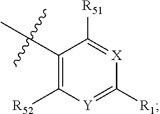

X is N or CH; Y is CR₄; R₄ is CH₂F, CHF₂, or CF₃; and R₁ is F or Cl. In a further embodiment, R₄ is CF₃; and R₁ is F or Cl. In a further embodiment, R₁ is F. In another further embodiment, R₁ is Cl.

In one embodiment, Z is

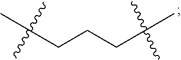 [SMILES: *CCC*]

### Q is

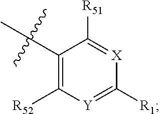

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In another further embodiment, R₁ is methyl substituted with 1, 2, or 3 fluorine atoms or methoxy substituted with 0, 1, 2, or 3 fluorine atoms. In a further embodiment, R₁ is OCHF₂ or OCF₃; X is N; and Y is CH. In another further embodiment, R₁ is CF₃; X is N; and Y is N.

In one embodiment, Z is

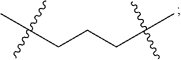 [SMILES: *CCC*]

### Q is

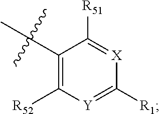

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In a further embodiment, R₄ is CH₂F, CHF₂, or CF₃. In a further embodiment, X is CH; Y is CR₄; R₁ is Cl; and R₄ is CF₃.

In one embodiment, Z is

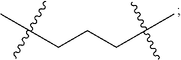 [SMILES: *CCC*]

### Q is

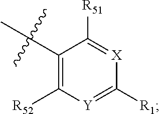

and R₅₁ and R₅₂ are each H. In another embodiment, one of R₅₁ and R₅₂ is H, and the other is F or Cl. In a further embodiment, one of R₅₁ and R₅₂ is H, and the other is F. In a further embodiment, X is CH; Y is CR₄; and R₄ is CF₃.

In one embodiment, Z is

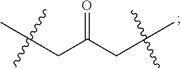 [SMILES: *CC(=O)C*]

### Q is

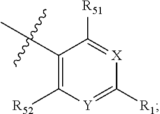

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In another embodiment, R₁ is methyl substituted with 1, 2, or 3 fluorine atoms or methoxy substituted with 0, 1, 2, or 3 fluorine atoms. In a further embodiment, R₁ is OCHF₂ or OCF₃; X is N; and Y is CH. In a further embodiment, R₁ is CF₃; X is N; and Y is N.

In one embodiment, Z is

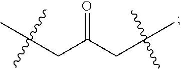 [SMILES: *CC(=O)C*]

### Q is

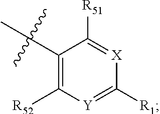

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In a further embodiment, R₄ is CH₂F, CHF₂, or CF₃. In a further embodiment, X is CH; Y is CR₄; R₁ is Cl; and R₄ is CF₃.

In one embodiment, Z is

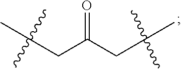 [SMILES: *CC(=O)C*]

### Q is

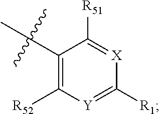

and R₅₁ and R₅₂ are each H. In another embodiment, one of R₅₁ and R₅₂ is H, and the other is F or Cl. In a further embodiment, one of R₅₁ and R₅₂ is H, and the other is F. In a further embodiment, X is CH; Y is CR₄; and R₄ is CF₃.

In one embodiment, Z is

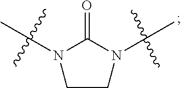 [SMILES: *N1CCN(*)C1=O]

### Q is

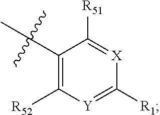

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In another embodiment, R₁ is methyl substituted with 1, 2, or 3 fluorine atoms or methoxy substituted with 0, 1, 2, or 3 fluorine atoms. In a further embodiment, R₁ is OCHF₂ or OCF₃; X is N; and Y is CH. In a further embodiment, R₁ is CF₃; X is N; and Y is N.

In one embodiment, Z is

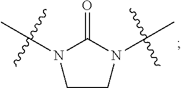 [SMILES: *N1CCN(*)C1=O]

### Q is

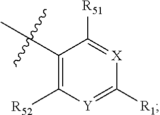

and R₁ is Cl, F, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms. In a further embodiment, R₁ is Cl or F. In a further embodiment, R₄ is CH₂F, CHF₂, or CF₃. In a further embodiment, X is CH; Y is CR₄; R₁ is Cl; and R₄ is CF₃.

In one embodiment, Z is

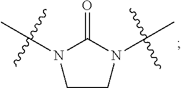 [SMILES: *N1CCN(*)C1=O]

### Q is

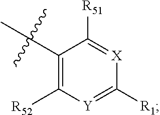

and R₅₁ and R₅₂ are each H. In another embodiment, one of R₅₁ and R₅₂ is H, and the other is F or Cl. In a further embodiment, one of R₅₁ and R₅₂ is H, and the other is F. In a further embodiment, X is CH; Y is CR₄; and R₄ is CF₃.

In one embodiment, a compound of Formula (Ib) is of Formula (Ib1):

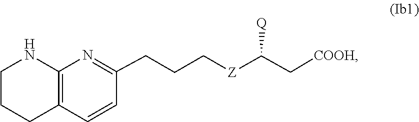

or a pharmaceutically acceptable salt or solvate thereof, wherein each of the variables is as defined above in Formula (Ib). Compounds of the present application include compounds of Formula (Ib1), wherein the variables and combinations thereof are illustrated in the various embodiments of Formula (Ib) above.

In one embodiment, a compound of Formula (Ib) is of Formula (Ib2) or (Ib3):

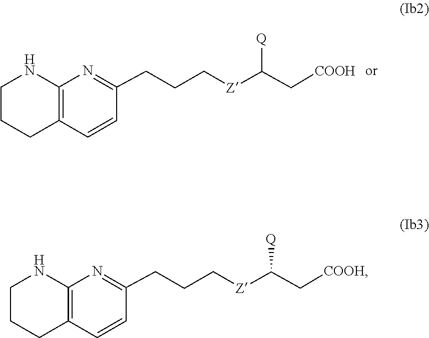

or a pharmaceutically acceptable salt or solvate thereof, wherein Z′ is

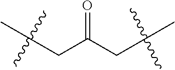 [SMILES: *CC(=O)C*]

and each of the other variables is as defined above in Formula (Ib). Compounds of the present application include compounds of Formula (Ib2) or (Ib3), wherein the variables and combinations thereof are illustrated in the various embodiments of Formula (Ib) above.

In one embodiment, Q is

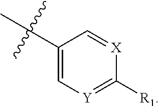

In one embodiment, X is N and Y is CR₄. In another embodiment, X and Y are each CR₄. In another embodiment, X and Y are each N.

In one embodiment, at least one R₄ is H. In one embodiment, at least one R₄ is CH₂F, CHF₂, or CF₃. In a further embodiment, at least one R₄ is CF₃.

In one embodiment, a compound of Formula (Ib) is of Formula (Ib4) or (Ib5):

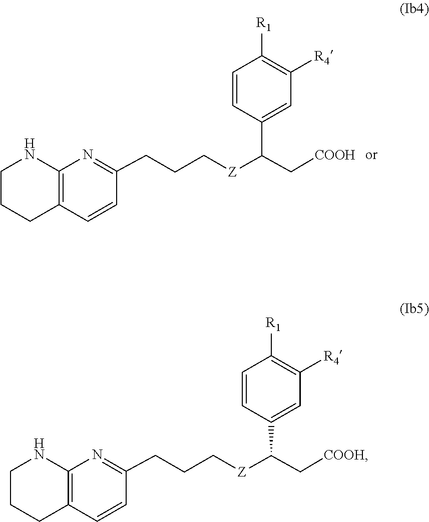

or a pharmaceutically acceptable salt or solvate thereof, wherein R₄′ is CH₂F, CHF₂, or CF₃, and each of the other variables are as defined above in Formula (Ib). Compounds of the present application include compounds of Formula (Ib4) or (Ib5), wherein the variables and combinations thereof are illustrated in the various embodiments of Formula (Ib) above.

In one embodiment, R₁ is H, F, or Cl. In a further embodiment, R₁ is F or Cl. R₁ is Cl.

In one embodiment, R₄′ is CF₃.

The compounds of the present application contain at least one fluorine atom. In one embodiment, the compounds of the present application contain at least one fluorine atom in the R₁ substituent. In another embodiment, the compounds of the present application contain at least one fluorine atom in the R₂ or R₃ substituent. In another embodiment, the compounds of the present application contain at least one fluorine atom in the R₄ substituent.

Representative compounds of the present application include the compounds listed in Tables 1a and 1b.

TABLE la

| Cmpd # | Chemical Structure | Cmpd # | Chemical Structure |
|---|---|---|---|
| A1 | 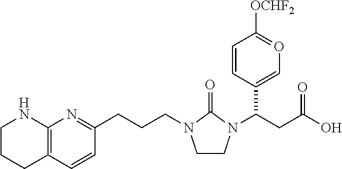 | A8 | 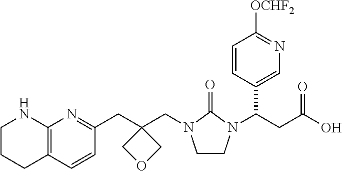 [SMILES: O=C(O)C[C@@H](c1ccc(OC(F)F)nc1)N1CCN(CC2(Cc3ccc4c(n3)NCCC4)COC2)C1=O] |
| A2 | 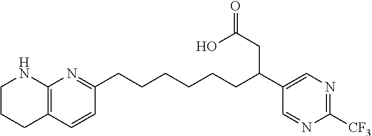 [SMILES: O=C(O)CC(CCCCCCc1ccc2c(n1)NCCC2)c1cnc(C(F)(F)F)nc1] | A9 | 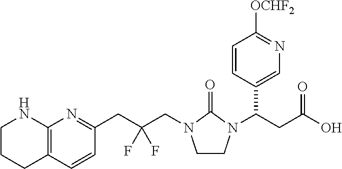 [SMILES: O=C(O)C[C@@H](c1ccc(OC(F)F)nc1)N1CCN(CC(F)(F)Cc2ccc3c(n2)NCCC3)C1=O] |
| A3 | 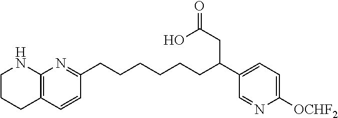 [SMILES: O=C(O)CC(CCCCCCc1ccc2c(n1)NCCC2)c1ccc(OC(F)F)nc1] | A10 | 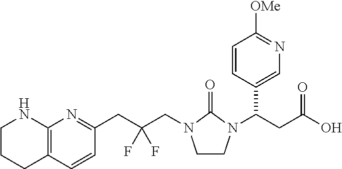 [SMILES: COc1ccc([C@H](CC(=O)O)N2CCN(CC(F)(F)Cc3ccc4c(n3)NCCC4)C2=O)cn1] |
| A4 | 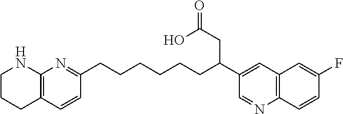 [SMILES: O=C(O)CC(CCCCCCc1ccc2c(n1)NCCC2)c1cnc2ccc(F)cc2c1] | A11 | 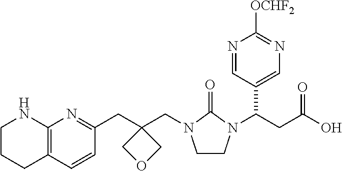 [SMILES: O=C(O)C[C@@H](c1cnc(OC(F)F)nc1)N1CCN(CC2(Cc3ccc4c(n3)NCCC4)COC2)C1=O] |
| A5 | 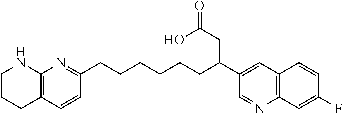 [SMILES: O=C(O)CC(CCCCCCc1ccc2c(n1)NCCC2)c1cnc2cc(F)ccc2c1] | A12 | 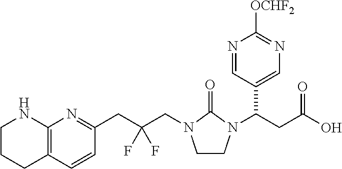 [SMILES: O=C(O)C[C@@H](c1cnc(OC(F)F)nc1)N1CCN(CC(F)(F)Cc2ccc3c(n2)NCCC3)C1=O] |
| A6 | 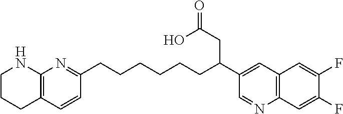 [SMILES: O=C(O)CC(CCCCCCc1ccc2c(n1)NCCC2)c1cnc2cc(F)c(F)cc2c1] | A13 | 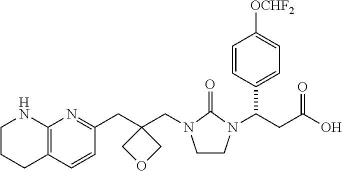 [SMILES: O=C(O)C[C@@H](c1ccc(OC(F)F)cc1)N1CCN(CC2(Cc3ccc4c(n3)NCCC4)COC2)C1=O] |
| A7 | 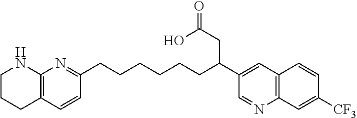 [SMILES: O=C(O)CC(CCCCCCc1ccc2c(n1)NCCC2)c1cnc2cc(C(F)(F)F)ccc2c1] | A14 | 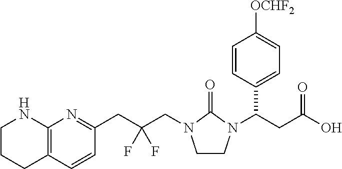 [SMILES: O=C(O)C[C@@H](c1ccc(OC(F)F)cc1)N1CCN(CC(F)(F)Cc2ccc3c(n2)NCCC3)C1=O] |

TABLE 1b

| Cmpd # | Chemical Structure | Cmpd # | Chemical Structure |
|---|---|---|---|
| A15 | 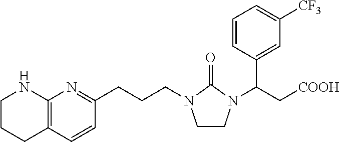 [SMILES: O=C(O)CC(c1cccc(C(F)(F)F)c1)N1CCN(CCCc2ccc3c(n2)NCCC3)C1=O] | A15s | 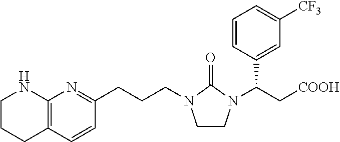 [SMILES: O=C(O)C[C@@H](c1cccc(C(F)(F)F)c1)N1CCN(CCCc2ccc3c(n2)NCCC3)C1=O] |
| A16 | 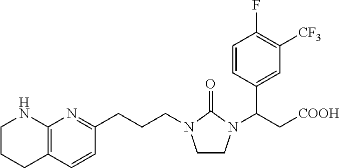 [SMILES: O=C(O)CC(c1ccc(F)c(C(F)(F)F)c1)N1CCN(CCCc2ccc3c(n2)NCCC3)C1=O] | A16s | 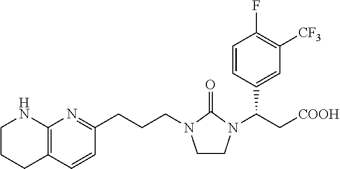 [SMILES: O=C(O)C[C@@H](c1ccc(F)c(C(F)(F)F)c1)N1CCN(CCCc2ccc3c(n2)NCCC3)C1=O] |
| A17 | 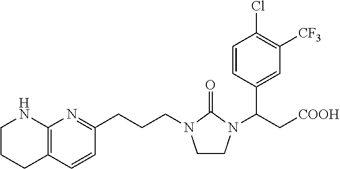 [SMILES: O=C(O)CC(c1ccc(Cl)c(C(F)(F)F)c1)N1CCN(CCCc2ccc3c(n2)NCCC3)C1=O] | A17s | 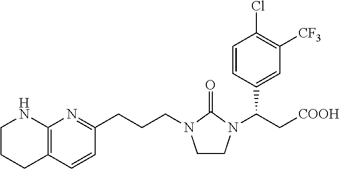 [SMILES: O=C(O)C[C@@H](c1ccc(Cl)c(C(F)(F)F)c1)N1CCN(CCCc2ccc3c(n2)NCCC3)C1=O] |
| A18 | 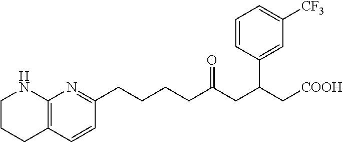 [SMILES: O=C(O)CC(CC(=O)CCCCc1ccc2c(n1)NCCC2)c1cccc(C(F)(F)F)c1] | A18s | 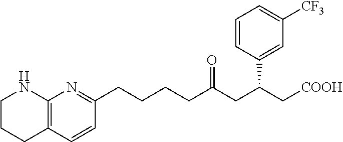 [SMILES: O=C(O)C[C@H](CC(=O)CCCCc1ccc2c(n1)NCCC2)c1cccc(C(F)(F)F)c1] |
| A19 | 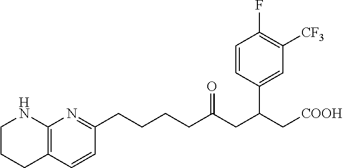 [SMILES: O=C(O)CC(CC(=O)CCCCc1ccc2c(n1)NCCC2)c1ccc(F)c(C(F)(F)F)c1] | A19s | 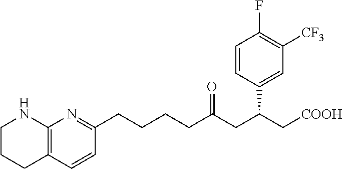 [SMILES: O=C(O)C[C@H](CC(=O)CCCCc1ccc2c(n1)NCCC2)c1ccc(F)c(C(F)(F)F)c1] |
| A20 | 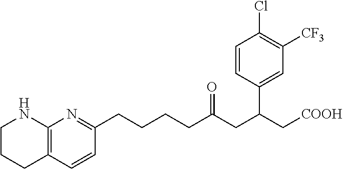 [SMILES: O=C(O)CC(CC(=O)CCCCc1ccc2c(n1)NCCC2)c1ccc(Cl)c(C(F)(F)F)c1] | A20s | 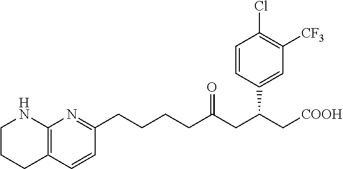 [SMILES: O=C(O)C[C@H](CC(=O)CCCCc1ccc2c(n1)NCCC2)c1ccc(Cl)c(C(F)(F)F)c1] |
| A21 | 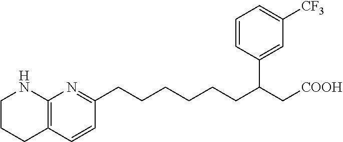 [SMILES: O=C(O)CC(CCCCCCc1ccc2c(n1)NCCC2)c1cccc(C(F)(F)F)c1] | A21s | 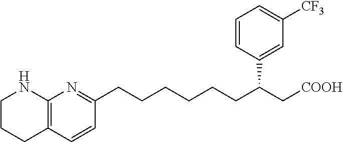 [SMILES: O=C(O)C[C@H](CCCCCCc1ccc2c(n1)NCCC2)c1cccc(C(F)(F)F)c1] |
| A21-1 enan- tiomer l | 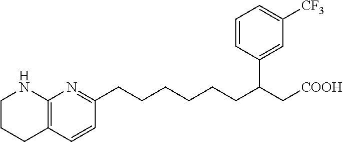 [SMILES: O=C(O)CC(CCCCCCc1ccc2c(n1)NCCC2)c1cccc(C(F)(F)F)c1] | A21-2 enan- tiomer 2 | 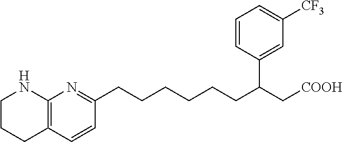 [SMILES: O=C(O)CC(CCCCCCc1ccc2c(n1)NCCC2)c1cccc(C(F)(F)F)c1] |
| A22 | 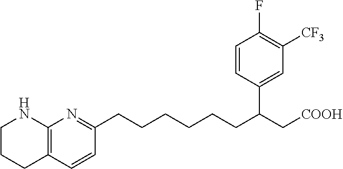 [SMILES: O=C(O)CC(CCCCCCc1ccc2c(n1)NCCC2)c1ccc(F)c(C(F)(F)F)c1] | A22s | 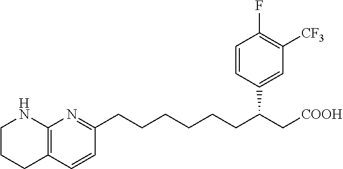 [SMILES: O=C(O)C[C@H](CCCCCCc1ccc2c(n1)NCCC2)c1ccc(F)c(C(F)(F)F)c1] |
| A23 | 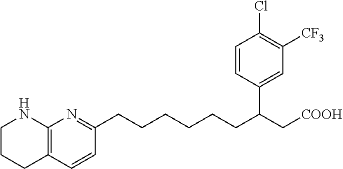 [SMILES: O=C(O)CC(CCCCCCc1ccc2c(n1)NCCC2)c1ccc(Cl)c(C(F)(F)F)c1] | A23s | 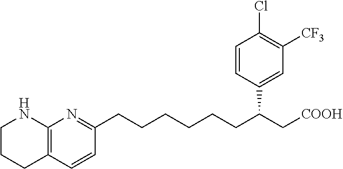 [SMILES: O=C(O)C[C@H](CCCCCCc1ccc2c(n1)NCCC2)c1ccc(Cl)c(C(F)(F)F)c1] |
| A24 | 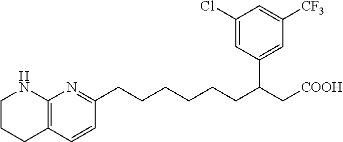 [SMILES: O=C(O)CC(CCCCCCc1ccc2c(n1)NCCC2)c1cc(Cl)cc(C(F)(F)F)c1] | A24s | 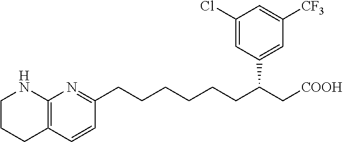 [SMILES: O=C(O)C[C@H](CCCCCCc1ccc2c(n1)NCCC2)c1cc(Cl)cc(C(F)(F)F)c1] |
| A25 | 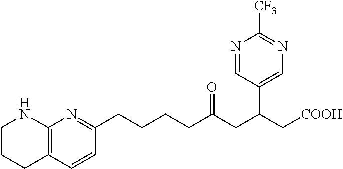 [SMILES: O=C(O)CC(CC(=O)CCCCc1ccc2c(n1)NCCC2)c1cnc(C(F)(F)F)nc1] | A25s | 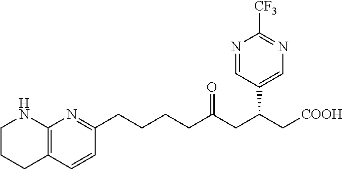 [SMILES: O=C(O)C[C@H](CC(=O)CCCCc1ccc2c(n1)NCCC2)c1cnc(C(F)(F)F)nc1] |
| A26 | 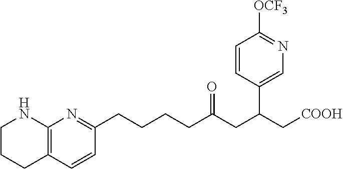 [SMILES: O=C(O)CC(CC(=O)CCCCc1ccc2c(n1)NCCC2)c1ccc(OC(F)(F)F)nc1] | A26s | 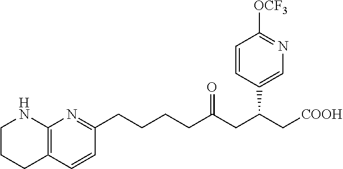 [SMILES: O=C(O)C[C@H](CC(=O)CCCCc1ccc2c(n1)NCCC2)c1ccc(OC(F)(F)F)nc1] |
| A27 | 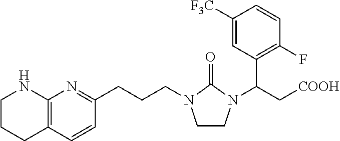 [SMILES: O=C(O)CC(c1cc(C(F)(F)F)ccc1F)N1CCN(CCCc2ccc3c(n2)NCCC3)C1=O] | A27s | 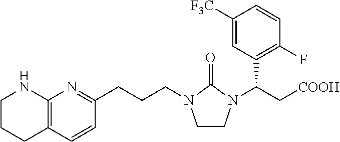 [SMILES: O=C(O)C[C@@H](c1cc(C(F)(F)F)ccc1F)N1CCN(CCCc2ccc3c(n2)NCCC3)C1=O] |
| A28 | 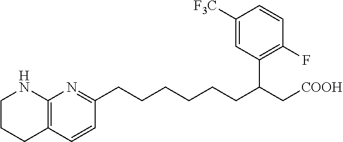 [SMILES: O=C(O)CC(CCCCCCc1ccc2c(n1)NCCC2)c1cc(C(F)(F)F)ccc1F] | A28s | 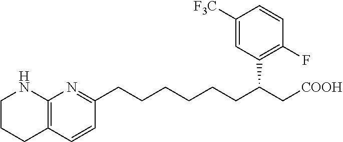 [SMILES: O=C(O)C[C@H](CCCCCCc1ccc2c(n1)NCCC2)c1cc(C(F)(F)F)ccc1F] |
| A29 | 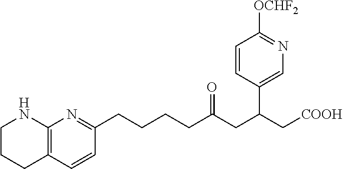 [SMILES: O=C(O)CC(CC(=O)CCCCc1ccc2c(n1)NCCC2)c1ccc(OC(F)F)nc1] | A29s | 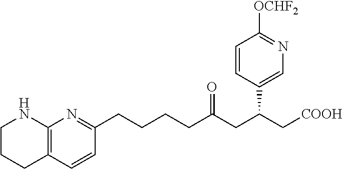 [SMILES: O=C(O)C[C@H](CC(=O)CCCCc1ccc2c(n1)NCCC2)c1ccc(OC(F)F)nc1] |
| A30 | 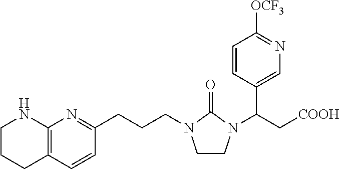 [SMILES: O=C(O)CC(c1ccc(OC(F)(F)F)nc1)N1CCN(CCCc2ccc3c(n2)NCCC3)C1=O] | A30s | 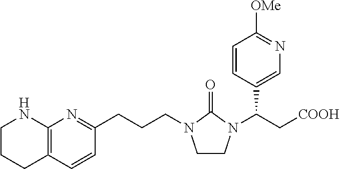 [SMILES: COc1ccc([C@H](CC(=O)O)N2CCN(CCCc3ccc4c(n3)NCCC4)C2=O)cn1] |

In one embodiment, the viral infection is an infection by a virus which binds to a cell surface integrin for attachment to or entry into the cell. In one embodiment, the viral infection is an infection by a virus which has a viral surface protein that binds or is capable of binding to a cell surface integrin. In one embodiment, the viral infection is an infection by a virus which has a viral surface protein that forms or is capable of forming a RGD motif. In one embodiment, the viral surface protein is a spike protein. In one embodiment, the viral infection is an infection by a virus which binds to a cell surface integrin for attachment to or entry into the cell, wherein the integrin binds or is capable of binding to a RGD motif.

In one embodiment, the viral infection is an infection by a virus (e.g., a virus which binds to a cell surface integrin for attachment to or entry into the cell, which has a viral surface protein that binds or is capable of binding to a cell surface integrin, and/or which has a viral surface protein that forms or is capable of forming a RGD motif) selected from human adenovirus, human cytomegalovirus, Kaposi's sarcoma-associated herpesvirus, Epstein-Barr virus, human immunodeficiency virus, UPS-associated hantaviruses, Sin Nombre virus, rotavirus, echovirus, foot-and-mouth disease virus, coxsackievirus, West Nile virus, Ebola virus, Ross River virus, human papillomavirus, and coronavirus. In one embodiment, the viral is selected from human adenovirus type ⅖, human cytomegalovirus, Kaposi's sarcoma-associated herpesvirus, Epstein-Barr virus, human immunodeficiency virus 1, UPS-associated hantaviruses NY-1 virus, Sin Nombre virus, rotavirus, echovirus types 1, echovirus types 9, foot-and-mouth disease virus, coxsackievirus A9, West Nile virus, Ebola virus, Ross River virus, human papillomavirus, and coronavirus. In one embodiment, the viral is coronavirus. In one embodiment, the coronavirus is selected from SARS-CoV, MERS-CoV, and SARS-CoV-2. In one embodiment, the coronavirus is SARS-CoV-2.

In one embodiment, the integrin binds or is capable of binding to a RGD motif (i.e., RGD binding integrin). In one embodiment, the integrin is selected from αVβ1, αVβ3, αVβ5, αVβ6, αVβ8, α5β1, α8β1, αLβ2, αMβ2, α2β1, α3β1, α6β1, α9β1, α4β1, α4β7, αIIα3, αxβ2, α1β1, α6β4, and α5β1. In one embodiment, the integrin is selected from αVβ1, αVβ3, αVβ5, αVβ6, αVβ8, α5β1, α8β1, α2β1, α3β1, α6β1, α9β1, α4β1, and α407. In one embodiment, the integrin is selected from αVβ1, αVβ3, αVβ5, αVβ6, αVβ8, α5β1, α8β1, and α2β1. In one embodiment, the integrin is selected from αVβ1, αVβ3, αVβ5, αVβ6, αVβ8, α5β1, and α8βl. In one embodiment, the integrin is selected from αVβ1, αVβ3, αVβ5, αVβ6, and αVβ8. In one embodiment, the integrin is selected from αVβ1 (e.g., ITGAV:ITGB1), αVβ3 (e.g., ITGAV:ITGB3), αVβ5 (e.g., ITGAV:ITGB5), αVβ6 (e.g., ITGAV:ITGB6), αVβ8 (e.g., ITGAV:ITGB8), α5β1 (e.g., ITGA5:ITGB1), and α8β1 (e.g., ITGA8:ITGB1).

The emergence of integrin-binding RGD domains near the distal tip of the SARS-Cov-2 spike protein, which are structurally similar to known viral integrin-binding proteins, such as foot and mouth disease virus capsid protein and African horse sickness virus, is considered to contribute to SARS-Cov-2 cellular binding and infection (Makowski, L. et al. Viruses, 2021, 13, 146). In addition, it is shown that αVβ3 and αVβ6 are bound by SARS-Cov-2 spike protein in an RGD dependent manner (Calver, J. et al. Thorax, 2021, 13, A22-A23). Accordingly, without wishing to be bound by the theory, although the level of ACE2 (which is considered to be the primary receptor for SARS-Cov-2 infection) is observed to be low in lung tissue in patients with high viral load, integrins such as αVβ3 and αVβ5 are upregulated in SARS-Cov-2-infected alveolar tissue (Calver, J. et al. Thorax, 2021, 13, A22-A23), and may contribute to viral entry and infection. Thus, inhibitors of RGD-binding integrins, such as the compounds of the present application, offer effective therapies for the treatment and prevention of SARS-Cov-2.

In one embodiment, an integrin antagonist (or integrin inhibitor) of the present application is an antagonist (or inhibitor) of an integrin that binds or is capable of binding to a RGD motif (i.e., RGD binding integrin). In one embodiment, the integrin is selected from αVβ1, αVβ3, αVβ5, αVβ6, αVβ8, α5β1, α8β1, αLβ2, αMβ2, α2β1, α3β1, α6β1, α9β1, α4β1, α407, αIIbβ3, αxβ2, all, α604, and α5β1. In one embodiment, the integrin is selected from αVβ1, αVβ3, αVβ5, αVβ6, αVβ8, α5β1, α8β1, α2β1, α3β1, α6β1, α9β1, α4β1, and α4β7. In one embodiment, the integrin is selected from αVβ1, αVβ3, αVβ5, αVβ6, αVβ8, α5β1, α8β1, and α2β1. In one embodiment, the integrin is selected from αVβ1, αVβ3, αVβ5, αVβ6, αVβ8, α5β1, and α8β1. In one embodiment, the integrin is selected from αVβ1, αVβ3, αVβ5, αVβ6, and αVβ8. In one embodiment, the integrin is selected from αVβ1 (e.g., ITGAV:ITGB1), αVβ3 (e.g., ITGAV:ITGB3), αVβ5 (e.g., ITGAV:ITGB5), αVβ6 (e.g., ITGAV:ITGB6), αVβ8 (e.g., ITGAV:ITGB8), α501 (e.g., ITGA5:ITGB1), and α801 (e.g., ITGA8:ITGB1).

In one embodiment, the condition or symptom associated with the viral infection, e.g., an infection by a virus described herein, is associated with an inflammatory or immunological response to the viral infection. In one embodiment, the condition or symptom is selected from fever, chill, cough, sore throat, shortness of breath, anosmia (i.e., loss of smell), diarrhea, nausea/vomiting, skin rashes, muscle pain, stomach pain, headache, pneumonia, kidney failure, thrombosis, organ failure, COVID-19, acute respiratory distress syndrome (ARDS), and severe acute respiratory syndrome (SARS). In one embodiment, the condition or symptom is ARDS or SARS.

In one embodiment, the inflammatory or immunological response includes increased cytokine and/or chemokine production. In one embodiment, the inflammatory or immunological response includes increased cytokine and/or chemokine production related to propagation and/or migration of NK cells, macrophages, plasmacytoid dendritic cells, and/or T cells. In one embodiment, the inflammatory or immunological response includes increased secretion or expression and/or elevated plasma level of one or more of IL-1β, IFN-γ, IP-10, MCP-1, IL-2, IL-4, IL-5, IL-6, IL-7, IL-1β, GCSF, MCP-1, MIP-1A, TNF-α, TNF-β, CXCL9, CXCL10, CCL2, CCL3, CCL5, CXCR3, CCR2, and/or CCR5.

In one embodiment, the use, compound for use, or method of the present application decreases or inhibits the inflammatory or immunological response, such as those described herein. In one embodiment, the use, compound for use, or method of the present application decreases or inhibits cytokine and/or chemokine production, and/or propagation and/or migration of NK cells, macrophages, plasmacytoid dendritic cells, and/or T cells. In one embodiment, the use, compound for use, or method of the present application decreases or inhibits secretion or expression and/or elevated plasma level of IL-1β, IFN-γ, IP-10, MCP-1, IL-2, IL-4, IL-5, IL-6, IL-7, IL-1β, GCSF, MCP-1, MIP-1A, TNF-α, TNF-β, CXCL9, CXCL10, CCL2, CCL3, CCL5, CXCR3, CCR2, and/or CCR5.

In one embodiment, the use, compound for use, or method of the present application further comprises administration of a second therapeutic agent. In one embodiment, the second therapeutic agent is selected from an anti-viral agent, an immune-suppressive agent, an anti-thrombotic agent, an anti-coagulant, an antibiotics, and other therapeutic agents that are capable of treating or preventing a viral infection or a condition or symptom associated with the viral infection. In one embodiment, the second therapeutic agent inhibits an RGD-binding integrin, such as an RGD-binding integrin described herein. In one embodiment, the second therapeutic agent is selected from cyclic penta-peptide inhibitor, cyclo-RGDfV, cilengetide, and the non-peptide integrin antagonist (e.g., JNJ-26076713 and EMD478761).

In one embodiment, the second therapeutic agent is an anti-viral agent. In one embodiment, the anti-viral agent is selected from remdesivir, LPV/r (lopinavir/ritonavir) (Kaletra), azidothymidine (AZT), ritonavir/lopinavir, azuvudine, danoprevir/ritonavir, sofosbuvir, darunavir, triazavirin, baloxivir, and clevudine.

In one embodiment, the second therapeutic agent is an immune-suppressive agent. In one embodiment, the immune-suppressive agent is selected from tocilizumab TCZ (Actemra), ixakizumab (Taltz), sarilumab, fosamprenavir (FVP), baricitinib (olumiant), hydroxychloroquine, chloroquine, anakinra, favipiravir, arbidol, ruxolitinib (Jakafi), sirolimus, siltuximab, mavrilimumab, emapalumab (Gamifant), dexamethasone, lefunomide, tofacitinib (Xeljanz), lenzilumab, clazakizumab, adalimumab, and mepolizumab.

In one embodiment, the second therapeutic agent is an anti-coagulant. In one embodiment, the anti-coagulant is defibrotide.

In one embodiment, the second therapeutic agent is an antibiotics. In one embodiment, the antibiotics is selected from azithromycin and carrimycin.

In one embodiment, the second therapeutic agent is a therapeutic agent that is capable of treating or preventing a viral infection or a condition or symptom associated with the viral infection. In one embodiment, the second therapeutic agent is selected from an H1 antagonist, convalescent plasma, a SGLT2 inhibitor, a BTK inhibitor, an anti-VEGF, a vasoactive peptide, a receptor tyrosine kinase inhibitor, and a thalidomide. In one embodiment, the second therapeutic agent is selected from ebastine, convalescent plasma, dapaglifozin, acalabrutinib, bevacizumab, ranibizumab, aflibercept, pembrolizumab, aviptadil, nintedanib, pirfenidone, and thalidomide.

In one embodiment, the second therapeutic agent is selected from an integrin antagonist, a cytotoxic/antiproliferative agent, an inhibitor of epidermal-derived, fibroblast-derived, or platelet-derived growth factor, an inhibitor of VEGF, an inhibitor of Flk-1/KDR, Flt-1, Tck/Tie-2, or Tic-1, and an inhibitor of phosphoinositide 3-kinase, and a mixture thereof. Nonlimiting examples of integrin antagonists are selected from (S)-2-((R)-2-((S)-2-((S)-2-((S)-1-acetylpyrrolidine-2-carboxamido)-3-(1H-imidazol-5-yl)propanamido)-3-hydroxypropanamido)-3-mercaptopropanamido) succinamide, and JSM6427. Nonlimiting examples of cytotoxic/antiproliferative agents are taxol, vincristine, vinblastine, and doxorubicin. Nonlimiting examples of inhibitors of epidermal-derived, fibroblast-derived, or platelet-derived growth factors are pazopanib, and sunitinib. Nonlimiting examples of inhibitors of vascular endothelial derived growth factor (VEGF) are bevacizumab and ranibizumab. Nonlimiting examples of inhibitors of phosphoinositide 3-kinase are indelalisib and 2-morpholin-4-yl-8-phenylchroman-4-one.

In one embodiment, the compound of the present application is Compound A1, A2, or A3, or a pharmaceutically acceptable salt or solvate thereof. In a further embodiment, the compound of the present application is Compound A1 or A2, or a pharmaceutically acceptable salt or solvate thereof. In a further embodiment, the compound of the present application is Compound A1, or a pharmaceutically acceptable salt or solvate thereof.

In one embodiment, a compound of the present application is a pharmaceutically acceptable salt. In one embodiment, a compound of the present application is a solvate. In a further embodiment, a compound of the present application is a hydrate.

In one embodiment, the present application provides use of Compound A1:

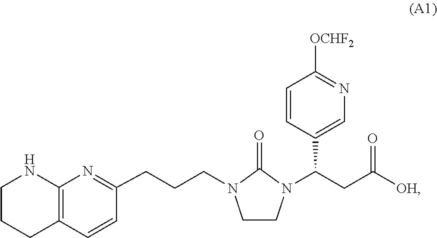 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, for treating or preventing a viral infection by SARS-CoV-2 or a condition or symptom associated with SARS-CoV-2 infection, such as COVID-19.

In one embodiment, the present application provides use of a pharmaceutical composition comprising Compound A1 or a pharmaceutically acceptable salt or solvate thereof, and a pharmaceutically acceptable carrier or excipient, for treating or preventing a viral infection by SARS-CoV-2 or a condition or symptom associated with SARS-CoV-2 infection, such as COVID-19.

In one embodiment, the present application provides use of Compound A1 or a pharmaceutically acceptable salt or solvate thereof, in combination with a second therapeutic agent, for treating or preventing a viral infection by SARS-CoV-2 or a condition or symptom associated with SARS-CoV-2 infection, such as COVID-19.

In one embodiment, the present application provides a method of treating or preventing a viral infection by SARS-CoV-2 or a condition or symptom associated with SARS-CoV-2 infection, such as COVID-19 in a subject in need thereof, comprising administering to the subject a therapeutically effective amount of Compound A1 or a pharmaceutically acceptable salt or solvate thereof.

In one embodiment, the present application provides a method of treating or preventing a viral infection by SARS-CoV-2 or a condition or symptom associated with SARS-CoV-2 infection, such as COVID-19 in a subject in need thereof, comprising administering to the subject a therapeutically effective amount of a pharmaceutical composition comprising Compound A1 or a pharmaceutically acceptable salt or solvate thereof, and a pharmaceutically acceptable carrier or excipient.

In one embodiment, the present application provides a method of treating or preventing a viral infection by SARS-CoV-2 or a condition or symptom associated with SARS-CoV-2 infection, such as COVID-19 in a subject in need thereof, comprising administering to the subject a therapeutically effective amount of Compound A1 or a pharmaceutically acceptable salt or solvate thereof, in combination with a second therapeutic agent.

In one embodiment, the present application provides use of Compound A1, or a pharmaceutically acceptable salt or solvate thereof, in the manufacture of a medicament for treating or preventing a viral infection by SARS-CoV-2 or a condition or symptom associated with SARS-CoV-2 infection, such as COVID-19.

In one embodiment, the present application provides use of a pharmaceutical composition comprising Compound A1 or a pharmaceutically acceptable salt or solvate thereof, and a pharmaceutically acceptable carrier or excipient, in the manufacture of a medicament for treating or preventing a viral infection by SARS-CoV-2 or a condition or symptom associated with SARS-CoV-2 infection, such as COVID-19.

In one embodiment, the present application provides use of Compound A1 or a pharmaceutically acceptable salt or solvate thereof, in combination with a second therapeutic agent, in the manufacture of a medicament for treating or preventing a viral infection by SARS-CoV-2 or a condition or symptom associated with SARS-CoV-2 infection, such as COVID-19.

In one embodiment, the present application provides Compound A1 or a pharmaceutically acceptable salt or solvate thereof, for the treatment or prevention of a viral infection by SARS-CoV-2 or a condition or symptom associated with SARS-CoV-2 infection, such as COVID-19.

In one embodiment, the present application provides a pharmaceutical composition comprising Compound A1 or a pharmaceutically acceptable salt or solvate thereof, and a pharmaceutically acceptable carrier or excipient, for the treatment or prevention of a viral infection by SARS-CoV-2 or a condition or symptom associated with SARS-CoV-2 infection, such as COVID-19.

In one embodiment, the present application provides Compound A1 or a pharmaceutically acceptable salt or solvate thereof, in combination with a second therapeutic agent, for the treatment or prevention of a viral infection by SARS-CoV-2 or a condition or symptom associated with SARS-CoV-2 infection, such as COVID-19.

In one embodiment, the present application provides Compound A1 or a pharmaceutically acceptable salt or solvate thereof, for use in the manufacture of a medicament for the treatment or prevention of a viral infection by SARS-CoV-2 or a condition or symptom associated with SARS-CoV-2 infection, such as COVID-19.

In one embodiment, the present application provides a pharmaceutical composition comprising Compound A1 or a pharmaceutically acceptable salt or solvate thereof, and a pharmaceutically acceptable carrier or excipient, for use in the manufacture of a medicament for the treatment or prevention of a viral infection by SARS-CoV-2 or a condition or symptom associated with SARS-CoV-2 infection, such as COVID-19.

In one embodiment, the present application provides Compound A1 or a pharmaceutically acceptable salt or solvate thereof, in combination with a second therapeutic agent, for use in the manufacture of a medicament for the treatment or prevention of a viral infection by SARS-CoV-2 or a condition or symptom associated with SARS-CoV-2 infection, such as COVID-19.

Compounds of the present application can be conveniently prepared by a variety of methods familiar to those skilled in the art, such as those described in WO 2014/124302 and WO 2016/134223.

The compounds of the present application may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers. It is intended that all of the possible optical isomers and diastereomers in mixtures and as pure or partially purified compounds are included within the ambit of the present application. The present application is meant to comprehend all such isomeric forms of these compounds.

If desired, racemic mixtures of the compounds may be separated so that the individual enantiomers are isolated. The separation can be carried out by methods well known in the art, such as contacting a racemic mixture of compounds with an enantiomerically pure compound to form a diastereomeric mixture, followed by separation of the individual diastereomers by standard methods, such as fractional crystallization or chromatography. The diasteriomeric mixture is often a mixture of diastereomeric salts formed by contacting a racemic mixture of compounds with an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which are well known in the art. Alternatively, any enantiomer of a compound may be obtained by stereoselective synthesis using optically pure starting materials or reagents of known configuration by methods well known in the art.

Some of the compounds of the present application may exist in unsolvated as well as solvated forms such as, for example, hydrates. “Solvate” means a solvent addition form that contains either a stoichiometric or non-stoichiometric amounts of the solvent molecules. Some compounds have a tendency to trap a fixed molar ratio of the solvent molecules in the crystalline solid state, thus forming a solvate. If the solvent is water, the solvate formed is a hydrate. When the solvent is alcohol, the solvate formed is an alcoholate. Hydrates are formed by the combination of one or more molecules of water with one of the substances (e.g., a compound of the present application) in which the water retains its molecular state as H₂O, such combination being able to form one or more hydrate. In hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates include sesquihydrates, monohydrates, dehydrates, and trihydrates. Equally suitable are the hydrates of salts of the compounds of the present application.

For use in medicine, the salts of the compounds of the present application refer to non-toxic “pharmaceutically acceptable salts”. Other salts may, however, be useful in the preparation of the compounds of the present application or pharmaceutically acceptable salts thereof. Salts encompassed within the term “pharmaceutically acceptable salts” refer to non-toxic salts of the compounds of the present application which can be prepared by reacting the free base with a suitable organic or inorganic acid. Representative salts include the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamottle (embonate), palmitate, pantothenate, phosphate/diphosphate, polygalacturonate, salicylate, stearate, sulfate, subacetate, succinate, tannate, tartrate, teoclate, tosylate, triethiodide, and valerate. Furthermore, where the compounds of the present application carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts which may be derived from ammonia or organic amines, such as, for example, diethylamine, triethylamine, ethyldiisopropylamine, procaine, dibenzylamine, N-methylmorpholine, dihydroabietylamine, or methylpiperidine.

The present application includes within its scope prodrugs of the compounds of the present application. In general, such prodrugs will be functional derivatives of the compounds of the present application which are readily convertible in vivo into the required compound. Thus, in the use, compound for use, or method of the present application, the term “administering” or “administration” shall encompass the treatment or prevention of the viral infection and various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of the present application into the biological milieu.

The present application also includes one or more metabolites of a compound of the present application.

The present application also comprehends deuterium labeled compounds of one of the Formulae described herein or the compounds listed in Tables 1a and 1b, wherein a hydrogen atom is replaced by a deuterium atom. The deuterium labeled compounds comprise a deuterium atom having an abundance of deuterium that is substantially greater than the natural abundance of deuterium, e.g., 0.015%. The term “deuterium enrichment factor” as used herein means the ratio between the deuterium abundance and the natural abundance of a deuterium. In one aspect, a compound of the present application has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). A compound of the present application or a pharmaceutically acceptable salt or solvate thereof that contains the aforementioned deuterium atom(s) is within the scope of the present application. Further, substitution with deuterium, i.e., ²H, can afford certain therapeutic advantages resulting from greater metabolic stability, for example, increased in vivo half-life and/or reduced dosage requirements.

The present application relates to pharmaceutical compositions comprising a compound of the present application as an active ingredient. In one aspect, the application provides a pharmaceutical composition comprising at least one compound of one of the formulae described herein, or a pharmaceutically acceptable salt or solvate thereof and one or more pharmaceutically acceptable carriers or excipients. In one aspect, the present application provides a pharmaceutical composition comprising at least one compound selected from Table 1a or 1b. In a further embodiment, the present application provides a pharmaceutical composition comprising at least one compound selected from Compounds A1, A2, and A3. In a further embodiment, the present application provides a pharmaceutical composition comprising at least one compound selected from Compounds A1 and A2. In a further embodiment, the present application provides a pharmaceutical composition comprising Compound A1.

As used herein, the term “composition” is intended to encompass a product comprising the specified ingredients in the specified amounts, as well as any product which results, directly or indirectly, from combination of the specified ingredients in the specified amounts.

A pharmaceutical compositions of the present application are formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The compounds of the present application can be formulated for oral administration in forms such as tablets, capsules (each of which includes sustained release or timed release formulations), pills, powders, granules, elixirs, tinctures, suspensions, syrups and emulsions. The compounds of the present application can also be formulated for intravenous (bolus or in-fusion), intraperitoneal, topical (e.g., ocular eye-drop), subcutaneous, intramuscular or transdermal (e.g., patch) administration, all using forms well known to those of ordinary skill in the pharmaceutical arts.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compound is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The compounds of the present application can be formulated for topical administration, such as solution, suspension, gel, oil, spray, drops, patches, cream, ointment, and lotion. The topic formulation of the present application may be in the form of an aqueous solution comprising an aqueous vehicle. The aqueous vehicle component of the ophthalmic formulation may comprise water and at least one pharmaceutically acceptable excipient. Suitable pharmaceutically acceptable excipients include those selected from the group consisting of a solubility enhancing agent, chelating agent, preservative, tonicity agent, viscosity/suspending agent, buffer, and pH modifying agent, and a mixture thereof.

Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated p-cyclodextrin (S—β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof. Preferably, solubility enhancing agent includes β-cyclodextrin sulfobutyl ether, hyrdoxypropyl-β-cyclodextrin, sulphated p-cyclodextrin (S-β-CD), and maltosyl-β-cyclodextrin, and mixtures thereof. The solubility enhancing agent(s) may be added in an amount of about 1 to about 20 wt %, preferably about 1 to about 10 wt %, and more preferably about 5 to about 10 wt %.

Examples of a chelating agent include those selected from the group consisting of ethylenediaminetetraacetic acid and metal salts thereof, disodium edetate, trisodium edetate, and tetrasodium edetate, and mixtures thereof. Disodium edetate is a particularly preferred chelating agent. The chelating agent(s) may be added in an amount of about 0.001 to about 0.05 wt %, preferably about 0.001 to about 0.02 wt %, more preferably about 0.002 to about 0.01 wt %, and most preferably about 0.002 to about 0.005 wt %.

Preferably, the aqueous vehicle includes a preservative. Preferred preservatives include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-β-hydroxybenzoate, sorbic acid, and mixtures thereof. More preferably, the preservative is a quaternary ammonium salt such as benzalkonium halides (preferably benzalkoniurn chloride), chlorhexidine gluconate, benzethonium chloride, cetyl pyridinium chloride, potassium sorbate, sodium benzoate, ethyl p-hydroxybenzoate, butyl p-hydroxybenzoate, or propylaminopropyl biguanide, or mixtures thereof. Propylaminopropyl biguanide is an especially preferred preservative. The preservative(s) may be used in an amount of about 0.00001 to about 0.0001 wt %, preferably about 0.00001 to about 0.00008 wt %, and more preferably about 0.00002 to about 0.00005 wt %.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. Preferably, the tonicity agent is selected from the group consisting of glycerin, mannitol, potassium chloride, and sodium chloride. More preferably mannitol and/or sodium chloride (and most preferably a mixture thereof) are employed. The tonicity agent(s) may be used in an amount of about 0.05 to about 8 wt %, preferably about 0.1 to about 6 wt %, more preferably about 0.1 to about 4 wt %, and most preferably about 0.2 to about 4 wt %.

The aqueous vehicle preferably also contains a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof. In preferred embodiments of the present application, the viscosity/suspending agent is a carbomer, more preferably Carbopol 974P. The viscosity/suspending agent(s) may be present in an amount of about 0.05 to about 2 wt %, preferably 0.1 to about 1 wt %, more preferably about 0.2 to about 0.8 wt %, and most preferably about 0.3 to about 0.5 wt %.

The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target ophthalmically acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof. The buffer agent(s) may be present in an amount of about 0.05 to about 5 wt %, preferably 0.1 to about 5 wt %, more preferably about 0.2 to about 5 wt %, and most preferably about 0.5 to about 5 wt %.

The topical formulation may further comprise a wetting agent. In any embodiment of the present application the wetting agent is preferably a non-ionic wetting agent. More preferably, the wetting agent is water soluble or swellable. Most preferably the wetting agent is water soluble. “Water soluble” is to be understood in the manner used in standard texts such as the “Handbook of Pharmaceutical Excipients” (Raymond C Rowe, Paul J Sheskey and Sian C Owen, Fifth Edition, Pharmaceutical Press and American Pharmacists Association 2006). Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof. Specific examples of suitable wetting agents include those selected from the group consisting of: polyoxyethylene-polyoxypropylene block copolymers (poloxamers) such as: polyoxyethylene (160) polyoxypropylene (30) glycol [Pluronic F68], polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Poloxamer 407, Pluronic F127], polyoxyethylene (20) polyoxypropylene (20) glycol [Pluronic L44], polyoxyethylenated sorbitan esters (polysorbates) such as poly(oxyethylene)sorbitan monopalmitate (polysorbate 40), poly(oxyethylene)sorbitan monostearate (polysorbate 60), poly(oxyethylene)sorbitan tristearate (polysorbate 65), poly(oxyethylene) sorbitan monooleate (polysorbate 80), poly(oxyethylene) sorbitan monolaurate, poly(oxyethylene) sorbitan trioleate, polyethoxylated ethers of castor oils such as polyoxyethylene hydrogenated castor oil 10, polyoxyethylene hydrogenated castor oil 40, polyoxyethylene hydrogenated castor oil 50 and polyoxyethylene hydrogenated castor oil 60, polyoxyl 40 stearate, sucrose fatty esters, and polyoxyethylene fatty esters, and mixtures thereof.

The second therapeutic agent can be administered via any administration routes, including oral administration in forms such as tablets, capsules (each of which includes sustained release or timed release formulations), pills, powders, granules, elixirs, tinctures, suspensions, syrups emulsions, intravenous administration (bolus or in-fusion), intraperitoneal administration, topical administration, subcutaneous administration, intramuscular administration, and transdermal (e.g., patch) administration.

Administration of the second therapeutic agent in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present application. “Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner.

In accordance with the method of the present application, the individual components of the combination can be administered separately at different times during the course of therapy or concurrently in divided or single combination forms. The present application is therefore to be understood as embracing all such regimens of simultaneous or alternating treatment, and the term “administering” is to be interpreted accordingly. It will be understood that the scope of combinations of the compounds of the present application with other agents useful for treating or prevent a viral infection or a condition or symptom associated therewith includes in principle any combination with any pharmaceutical composition useful for treating or preventing same.

The dosage regimen utilizing the compounds of the present application and that of the second therapeutic agent is selected in accordance with a variety of factors including type, species, age, weight, sex and medical condition of the patient; the severity of the condition to be treated; and the particular compound or salt thereof employed. An ordinary skilled physician, veterinarian or clinician can readily determine and prescribe the effective amount of the drug required to prevent, counter or arrest the progress of the condition or symptom.

In the methods of the present application, the compounds herein described in detail can form the active ingredient, and are typically administered in admixture with suitable pharmaceutical diluents, excipients or carriers (collectively referred to herein as “carrier”) suitably selected with respect to the intended topical administration to the eye and consistent with conventional pharmaceutical practices.

For purposes of the present application, the following definitions will be used (unless expressly stated otherwise):

“SARS” means Severe Acute Respiratory Syndrome, which is caused by or associate with infection by coronavirus. “SARS-CoV” means Severe Acute Respiratory Syndrome Coronavirus, “SARS-CoV-1” means Severe Acute Respiratory Syndrome Coronavirus 1, and “SARS-CoV-2” means Severe Acute Respiratory Syndrome Coronavirus 2. “COVID-19” means a respiratory illness caused by or associated with infection by SARS-CoV-2. “MERS” means Middle Eastern Respiratory Syndrome.

“Pharmaceutical” or “pharmaceutically acceptable” when used herein as an adjective, means substantially non-toxic and substantially non-deleterious to the recipient.

By “pharmaceutical composition” it is further meant that the carrier, diluent, solvent, excipient, and salt must be compatible with the active ingredient of the formulation (e.g., a compound of the present application). It is understood by those of ordinary skill in this art that the terms “pharmaceutical formulation” and “pharmaceutical composition” are generally interchangeable, and they are so used for the purposes of this application.

“Solution” refers to a clear, homogeneous liquid dosage form that contains one or more chemical substances dissolved in a solvent or mixture of mutually miscible solvents. Because molecules of a therapeutic agent substance in solution are uniformly dispersed, the use of solutions as dosage forms generally provides assurance of uniform dosage upon administration and good accuracy when the solution is diluted or otherwise mixed. “Solution” as disclosed herein contemplates any variations based on the current state of the art or variations achieved by one skilled in the art.

“Suspension” refers to a liquid dosage form that contains solid particles dispersed in a liquid vehicle. “Suspension” as disclosed herein contemplates any variations based on the current state of the art or variations achieved by one skilled in the art.

“Excipient” is used herein to include any other compound that is not a therapeutically or biologically active compound and may be contained in or combined with one or more of the compounds of the present application. As such, an excipient should be pharmaceutically or biologically acceptable or relevant (for example, an excipient should generally be non-toxic to the subject). “Excipient” includes a single such compound and is also intended to include a plurality of excipients. For the purposes of the present disclosure the term “excipient” and “carrier” are used interchangeably throughout the description of the present application.

“Therapeutically effective amount” refers to that amount of a drug or pharmaceutical agent that will elicit the biological or medical response of a tissue, system, animal, or human that is being sought by a researcher or clinician. For any compound, the therapeutically effective amount can be estimated initially either in cell culture assays, e.g., of cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

“Treat,” “treating,” or “treatment” refers to decreasing the symptoms, markers, and/or any negative effects of a disease or condition in any appreciable degree in a subject who currently has the disease or condition. In some embodiments, treatment may be administered to a subject who exhibits only early signs of a disease or condition for the purpose of decreasing the risk of developing the disease or condition. In some embodiments, “Treat,” “treating,” or “treatment” refers to amelioration of one or more symptoms of a disease or condition. For example, amelioration of one or more symptoms of a disease or condition includes a decrease in the severity, frequency, and/or length of one or more symptoms of a disease or condition.

“Prevent,” “prevention,” or “preventing” refers to any method to partially or completely prevent or delay the onset of one or more symptoms or features of a disease or condition. Prevention may be administered to a subject who does not exhibit any sign of a disease or condition.

“Subject” means a human or animal (in the case of an animal, more typically a mammal). In one aspect, the subject is a human.

The term “symptom” is defined as an indication of disease, illness, injury, or that something is not right in the body. Symptoms are felt or noticed by the individual experiencing the symptom, but may not easily be noticed by others. Others are defined as non-health-care professionals.

An “integrin antagonist” refers to a compound which binds to and inhibits or interferes with the function of an integrin. The compounds bind to the receptors as antagonists, blocking or interfering with the binding of a viral protein, such as a viral surface protein, while not provoking a biological response themselves.

“Alkyl” refers to straight chain or branched alkyl of the number of carbon atoms specified (e.g., C₁-C₄ alkyl), or any number within this range (methyl, ethyl, propyl, i-propyl, butyl, i-butyl, t-butyl, etc.).

“Alkoxy” refers to straight chain or branched alkoxides of the number of carbon atoms specified (e.g., C₁-C₆ alkoxy), or any number within this range (methoxy, ethoxy, propoxy, i-propoxy, butoxy, i-butoxy, t-butoxy, etc.).

“Carbocyclic ring” refers to saturated cycloalkyl of the number of carbon atoms specified (i.e., C₃ or C₄), such as cyclopropyl and cyclobutyl.

“Heterocyclic ring” refers to saturated heterocyclic ring of the number of carbon atoms specified (i.e., C₃ or C₄), further comprising one additional heteroatoms selected from N, O, and S.

The term “about” refers to a range of values which can be 15%, 10%, 8%, 5%, 3%, 2%, 1%, or 0.5% more or less than the specified value. For example, “about 10%” can be from 8.5% to 11.5%. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

### EXAMPLES

### Example 1. Coronavirus S-Protein Pseudotype Production

To produce seed particles (VSVΔG-luc/GFP+VSV-G), 293T cells were seeded in six-well plates and transfected 24 h later with 2 μg VSV-luc/GFP, 2 μg T7 polymerase, 0.5 μg VSV-N, 0.25 μg VSV-L, 1.25 μg VSV-P, and 1 μg VSV-G. VSV seed particles were harvested 48 h post-transfection. Cell supernatants were collected, cleared from cell debris by centrifugation, aliquoted, and stored at −80° C.

Coronavirus spike protein pseudotypes were produced according to methods described in Letko, M. et al. (Cell Rep. 24, 1730-1737 (2018)). 293T cells were seeded into six-well plates pre-coated with poly-l-lysine (Sigma-Aldrich) and transfected the next day with 1,200 ng of empty plasmid and 400 ng of plasmid encoding coronavirus spike or green fluorescent protein (GFP) as a no-spike control. After 24 h, transfected cells were infected with VSVΔG particles pseudotyped with VSV-G, as described in Takada, A. et al. (Proc. Natl Acad. Sci. USA 94, 14764-14769 (1997)). After 1 h of incubating at 37° C., cells were washed three times and incubated in 2 ml DMEM supplemented with 2% FBS, penicillin/streptomycin and 1-glutamine for 48 h. Supernatants were collected, centrifuged at 500 g for 5 min, aliquoted, and stored at −80° C.

### Example 2. Luciferase-Based Cell Entry Assay

Target cells were seeded in black 96-well plates and inoculated, in triplicate, with equivalent volumes of coronavirus S protein pseudotype stocks. For the trypsin experiments, pseudotype stocks were diluted 1:1 in DMEM without FBS, trypsin was added to a final concentration of 2,500 μg/ml and the samples were incubated at 37° C. for 15 min. The samples were then diluted again 1:1 in cold DMEM supplemented with 2% FBS, and added to cells. Inoculated plates were centrifuged at 1,200 g at 4° C. for 1 h and incubated overnight at 37° C.

Approximately 18-20 h post-infection, Bright-Glo luciferase reagent (Promega) was added to each well, 1:1, without removing the culture media, and luciferase was measured. Relative entry was calculated as the fold-entry over the negative control, by normalizing the relative light unit for S protein pseudotypes to the plate relative light unit average for the no-S protein control.

### Example 3. Western Blot

Producer cells (spike-transfected 293T) were lysed in 1% sodium dodecyl sulfate, 150 mM NaCl, 50 mM Tris-HCl, and 5 mM EDTA and clarified by centrifugation at 14,000 g for 20 min.

Pseudotyped particles were concentrated from producer cell supernatants that were overlaid on a 10% OptiPrep cushion in PBS (Sigma-Aldrich) and centrifuged at 20,000 g for 2 h at 4° C. Lysates and concentrated particles were analyzed for FLAG (Sigma-Aldrich; A8592; 1:10,000), GAPDH (Sigma-Aldrich; G8795; 1:10,000), and/or VSV-M (Kerafast; 23H12; 1:5,000) expression on 10% Bis-Tris PAGE gel (Thermo Fisher Scientific).

### Example 4. Ischemia-Reperfusion Lung Injury Model

Sprague-Dawley rats (300-500 g) (Charles River Laboratories, Wilmington, Mass.) were transtracheally intubated and ventilated under isoflurane anesthesia with a tidal volume of 6 ml/kg, positive end-expiratory pressure (PEEP) of 10 cm H₂O, and 100% oxygen (Model 683; Harvard Apparatus Co., Holliston, Mass.). A median sternotomy was performed, heparin (200 IU) was injected into the right ventricle, and cannulas were placed in the pulmonary artery and left ventricle. Unilateral lung IR was induced by right pulmonary artery ligation for 30 min followed by release and reperfusion for 3 h. Rats were treated with a compound of the present application immediately before the experiment.

Previous studies have determined this to be the peak time for soluble VEGFRII secretion from the liver³. Contralateral lungs served as non-ischemic controls.

### Example 5. Ventilation Induced Lung Injury Model

Mice were transtracheally intubated and ventilated with a high tidal volume of 20 ml/kg at a rate of 48 breaths/min (without PEEP) for 4 h using a mouse ventilator (Model 683; Harvard Apparatus Co). Animals were anesthetized using serial ketamine (37.5 mg/ml) and xylazine (250 mg/ml) intraperitoneal injections (100 ul/20 g) with equal volume injections of normal saline in matched animals. Matched non-ventilated mice were administered equal volumes of anesthesia and saline to serve as baseline controls. Mice were administered a compound of the present application 24 h before the experiment. TGF-Beta-RII-Ig (25 ug in 100 ul sterile saline) was administered intravenously immediately before initiation of ventilation. Lungs were harvested immediately after 4 h of ventilation for lung vascular leak assay preparation.

### Example 6. Quantification of VEGF-Induced Vascular Leak

Vascular leak was studied 7 d after administration of a compound of the present application, by measuring the extravasation of Evan's blue dye (30 mg/kg in 50 ul per mouse). After 5 min, vascular leak was induced by dermal injection of VEGF (100 ng in 10 ul normal saline) into mouse ears. After 1 h, 4 mm punch sections around the VEGF injection site were harvested and formamide-extracted dye was quantified as absorbance at 610 nm with a Spectra Max 190 Spectrophotometer (Molecular Devices, Sunnyvale, Calif.).

### Example 7. Lung Vascular Leak and Protein Permeability

0.5 uCi of ¹²⁵I-labeled albumin in 300 L sterile normal saline was administered intraperitoneally 4 h before lung harvest to ensure adequate distribution. After each experiment, a blood sample was obtained to measure the hemoglobin concentration and the water-to-dry weight ratio of blood for the extravascular plasma equivalents (EVPE) calculation.

Lungs (left for IR, and bilateral for VILI) were homogenized and the extravascular lung water determined by calculating the water-to-dry weight ratio using the following equation:W/D - Q_{wet}/Q_{dry}, in which Q_{wet} is the difference between the water content of the lung homogenate and the water content of the blood in the lung, and Q_{dry} is the dry lung weight calculated as the weight of the lungs minus the blood and water volumes in the lung. Lung endothelial permeability to albumin, expressed as EVPE in ml, calculated using the following equation: EVPE (CH−(CP_{end}QB))/CPₐᵥₑ. CH represents the ¹²⁵I counts/min/g in the homogenized lung, CP_{end} represents the counts/min/g in plasma at the end of the experiment, and CPₐᵥₑ represents the average counts/min/g in the plasma samples at the end of the experiment. QB is the blood volume in the lungs determined by the gravimetric method using weights from wet and dried lung homogenates (Frank, J. et al. (J Appl Physiol 89, 1255-1265 (2000), Pittet J. F. et al. (J. Clin. Invest. 94, 663-671 (1994)). Counts were measured on a Wizard gamma counter (Perkin-Elmer). Control lungs included the contralateral nonischemic, nonreperfused right lung for IR, and lungs from nonventilated mice for VILI. Baseline lungs for IR were harvested from animals not subject to pulmonary artery ligation.

### EQUIVALENTS

Those skilled in the art will recognize, or be able to ascertain using no more than routine experimentation, many equivalents to the specific embodiments and methods described herein. Such equivalents are intended to be encompassed by the scope of the present application.

All patents, patent applications, and literature references cited herein are hereby expressly incorporated by reference.

1. A method of treating or preventing a viral infection in a subject in need thereof, comprising administering to the subject a therapeutically effective amount of an integrin inhibitor represented by Formula (I): 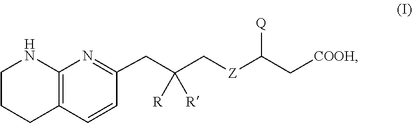
or a pharmaceutically acceptable salt or solvate thereof, wherein:
Z is 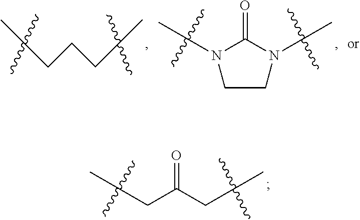 [SMILES: *CCC* ; *N1CCN(*)C1=O ; *CC(=O)C*]
Q is 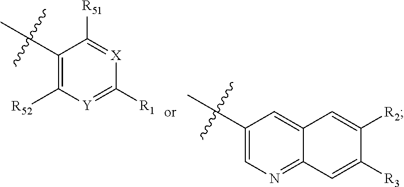
X is CR₄ or N;
Y is CR₄ or N;
R and R′ are each independently H or F, or R and R′, together with the carbon atom to which they are attached, form a 3—or 4-membered carbocyclic or heterocyclic ring;
R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;
R₂ and R₃ are each independently H, F, CH₂F, CHF₂, or CF₃, provided that one of R₂ and R₃ is not H;
each R₄ is independently H, CH₂F, CHF₂, or CF₃; and
R₅₁ and R₅₂ are each independently H, F, or Cl;
provided that the compound of Formula (I) contains at least one fluorine atom; and
wherein the viral infection is an infection by human adenovirus, human cytomegalovirus, Kaposi's sarcoma-associated herpesvirus, Epstein-Barr virus, human immunodeficiency virus, HPS-associated hantaviruses, Sin Nombre virus, rotavirus, echovirus, foot-and-mouth disease virus, coxsackievirus, West Nile virus, Ebola virus, Ross River virus, human papillomavirus, or coronavirus.

2. A method of treating or preventing a condition or symptom associated with a viral infection in a subject in need thereof, comprising administering to the subject a therapeutically effective amount of an integrin inhibitor represented by Formula (I): 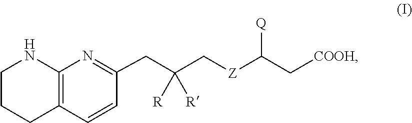
or a pharmaceutically acceptable salt or solvate thereof, wherein:
Z is 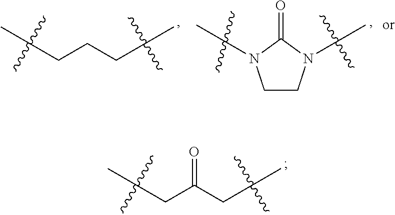 [SMILES: *CCC* ; *N1CCN(*)C1=O ; *CC(=O)C*]
Q is 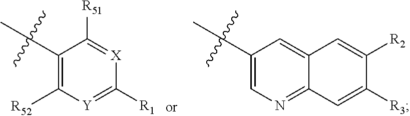
X is CR₄ or N;
Y is CR₄ or N:
R and R′ are each independently H or F, or R and R′, together with the carbon atom to which they are attached, form a 3—or 4-membered carbocyclic or heterocyclic ring;
R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;
R₂ and R₃ are each independently H, F, CH₂F, CHF₂, or CF₃, provided that one of R₂ and R₃ is not H;
each R₄ is independently H, CH₂F, CHF₂, or CF₃; and
R₅₁ and R₅₂ are each independently H, F, or Cl;
provided that the compound of Formula (I) contains at least one fluorine atom; and
wherein the viral infection is an infection by human adenovirus, human cytomegalovirus, Kaposi's sarcoma-associated herpesvirus, Epstein-Barr virus, human immunodeficiency virus, HPS-associated hantaviruses, Sin Nombre virus, rotavirus, echovirus, foot-and-mouth disease virus, coxsackievirus, West Nile virus, Ebola virus, Ross River virus, human papillomavirus, or coronavirus.

3. (canceled)

4. (canceled)

5. The method of claim 1, wherein the virus is selected from the group consisting of: human adenovirus, human cytomegalovirus, Kaposi's sarcoma-associated herpesvirus, Epstein-Barr virus, West Nile virus, Ebola virus, Ross River virus, human papillomavirus, and coronavirus.

6. The method of claim 5, wherein the virus is a coronavirus.

7. The method of claim 6, wherein the coronavirus is selected from SARS-CoV, MERS-CoV, and SARS-CoV-2.

8. The method of claim 7, wherein the coronavirus is SARS-CoV-2.

9. The method of claim 2, wherein the condition or symptom is an inflammatory or immunological response to the viral infection.

10. The method of claim 9, wherein the inflammatory or immunological response comprises increased cytokine and/or chemokine production.

11. The method of claim 9, wherein the inflammatory or immunological response is selected from increased secretion or expression and/or elevated plasma level of one or more of IL-1β, IFN-γ, IP-10, MCP-1, IL-2, IL-4, IL-5, IL-6, IL-7, IL-1β, GCSF, MCP-1, MIP-1A, TNF-α, TNF-β, CXCL9, CXCL10, CCL2, CCL3, CCL5, CXCR3, CCR2, and/or CCR5.

12. The method of claim 2, wherein the condition or symptom is selected from fever, chill, cough, sore throat, shortness of breath, anosmia, diarrhea, nausea/vomiting, skin rashes, muscle pain, stomach pain, headache, pneumonia, kidney failure, thrombosis, organ failure, COVID-19, acute respiratory distress syndrome (ARDS), and severe acute respiratory syndrome (SARS).

13. The method of claim 1, further comprising administering a second therapeutic agent.

14. The method of claim 13, wherein the second therapeutic agent is selected from an anti-viral agent, an immune-suppressive agent, an anti-thrombotic agent, an anti-coagulant, an antibiotics, and other therapeutic agents that are capable of treating or preventing a viral infection or a condition or symptom associated with the viral infection.

15. The method of claim 1, wherein the integrin inhibitor is a compound of Formula (I-1): 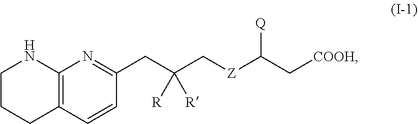
or a pharmaceutically acceptable salt thereof, wherein:
Z is 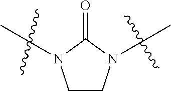 [SMILES: *N1CCN(*)C1=O]
Q is 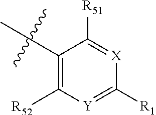
X is CR₄ or N;
Y is CR₄ or N;
R and R′ are each independently H or F, or R and R′, together with the carbon atom to which they are attached, form a 3—or 4-membered carbocyclic or heterocyclic ring;
R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;
each R₄ is independently H, CH₂F, CHF₂, or CF₃; and
R₅₁ and R₅₂ are each independently H, F, or Cl;
provided that the compound of Formula (I-1) contains at least one fluorine atom.

16. The method of claim 1, wherein the integrin inhibitor is a compound of Formula (Ia): 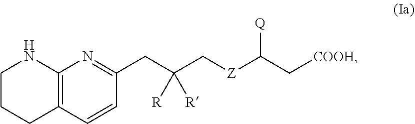
or a pharmaceutically acceptable salt or solvate thereof, wherein:
Z is 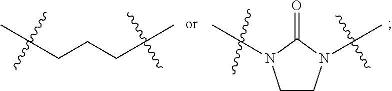 [SMILES: *CCC* ; *N1CCN(*)C1=O]
R and R′ are each independently H or F, or R and R′, together with the carbon atom to which they are attached, form a 3—or 4-membered carbocyclic or heterocyclic ring;
Q is 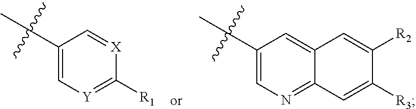
X is CH or N;
Y is CH or N;
R₁ is C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms; and
R₂ and R₃ are each independently H, F, CH₂F, CHF₂, or CF₃, provided that one of R₂ and R₃ is not H,
provided that the compound of Formula (Ia) contains at least one fluorine atom.

17. The method of claim 1, wherein the integrin inhibitor is a compound of Formula (Ib): 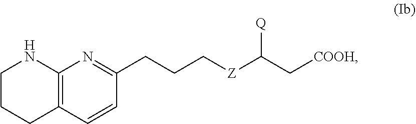
or a pharmaceutically acceptable salt or solvate thereof, wherein:
Z is 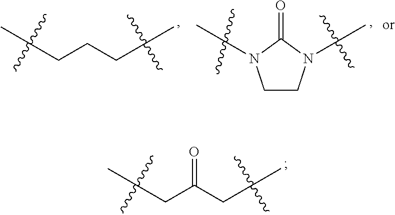 [SMILES: *CCC* ; *N1CCN(*)C1=O ; *CC(=O)C*]
Q is 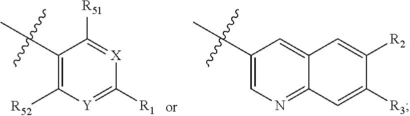
X is CR₄ or N;
Y is CR₄ or N;
R₁ is H, F, Cl, C₁-C₄ alkyl substituted with 1, 2, 3, 4, 5, 6, 7, 8, or 9 fluorine atoms, or C₁-C₆ alkoxy substituted with 0, 1, 2, 3, 4, 5, 6, or 7 fluorine atoms;
R₂ and R₃ are each independently H, F, CH₂F, CHF₂, or CF₃, provided that one of R₂ and R₃ is not H;
each R₄ is independently H, CH₂F, CHF₂, or CF₃; and
R₅₁ and R₅₂ are each independently H, F, or Cl;
provided that the compound of Formula (T) contains at least one fluorine atom, and provided that when Z is 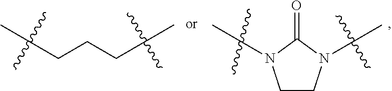 [SMILES: *CCC* ; *N1CCN(*)C1=O]
R₁ is not H, F, or Cl, and R₅₁ and R₅₂ are each H, then at least one of X and Y is CR₄, and R₄ is C₁H₂F, CHF₂, or CF₃.

18. The method of claim 1, wherein the integrin inhibitor is a compound selected from Table 1a or Table 1b or a pharmaceutically acceptable salt thereof:
TABLE 1a

| Cmpd No. | Chemical Structure | Cmpd No. | Chemical Structure |
|---|---|---|---|
| A1 | 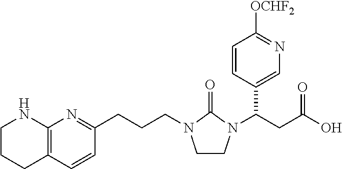 [SMILES: O=C(O)C[C@@H](c1ccc(OC(F)F)nc1)N1CCN(CCCc2ccc3c(n2)NCCC3)C1=O] | A8 | 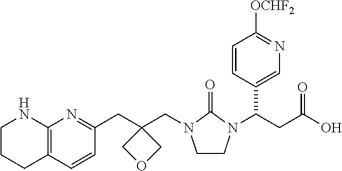 [SMILES: O=C(O)C[C@@H](c1ccc(OC(F)F)nc1)N1CCN(CC2(Cc3ccc4c(n3)NCCC4)COC2)C1=O] |
| A2 | 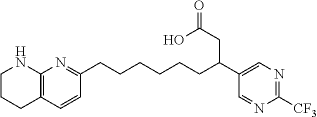 [SMILES: O=C(O)CC(CCCCCCc1ccc2c(n1)NCCC2)c1cnc(C(F)(F)F)nc1] | A9 | 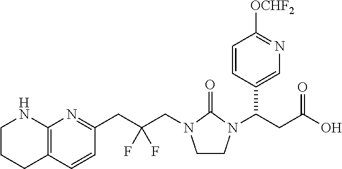 [SMILES: O=C(O)C[C@@H](c1ccc(OC(F)F)nc1)N1CCN(CC(F)(F)Cc2ccc3c(n2)NCCC3)C1=O] |
| A3 | 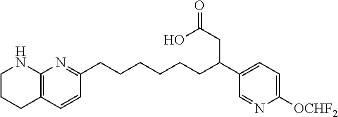 [SMILES: O=C(O)CC(CCCCCCc1ccc2c(n1)NCCC2)c1ccc(OC(F)F)nc1] | A10 | 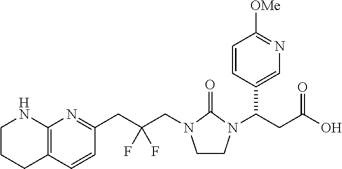 [SMILES: COc1ccc([C@H](CC(=O)O)N2CCN(CC(F)(F)Cc3ccc4c(n3)NCCC4)C2=O)cn1] |
| A4 | 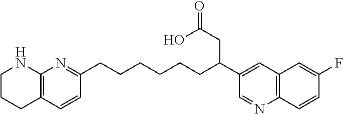 [SMILES: O=C(O)CC(CCCCCCc1ccc2c(n1)NCCC2)c1cnc2ccc(F)cc2c1] | A11 | 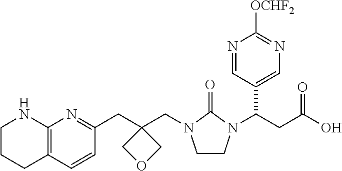 [SMILES: O=C(O)C[C@@H](c1cnc(OC(F)F)nc1)N1CCN(CC2(Cc3ccc4c(n3)NCCC4)COC2)C1=O] |
| A5 | 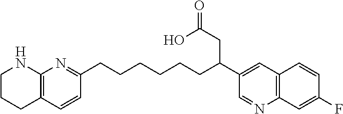 [SMILES: O=C(O)CC(CCCCCCc1ccc2c(n1)NCCC2)c1cnc2cc(F)ccc2c1] | A12 | 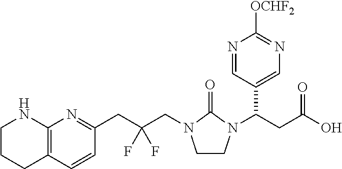 [SMILES: O=C(O)C[C@@H](c1cnc(OC(F)F)nc1)N1CCN(CC(F)(F)Cc2ccc3c(n2)NCCC3)C1=O] |
| A6 | 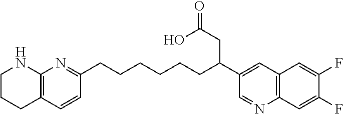 [SMILES: O=C(O)CC(CCCCCCc1ccc2c(n1)NCCC2)c1cnc2cc(F)c(F)cc2c1] | A13 | 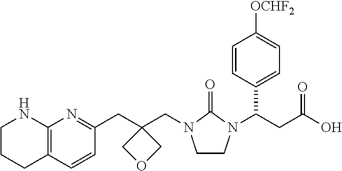 [SMILES: O=C(O)C[C@@H](c1ccc(OC(F)F)cc1)N1CCN(CC2(Cc3ccc4c(n3)NCCC4)COC2)C1=O] |
| A7 | 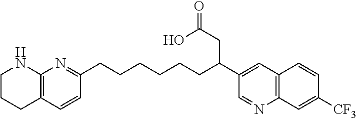 [SMILES: O=C(O)CC(CCCCCCc1ccc2c(n1)NCCC2)c1cnc2cc(C(F)(F)F)ccc2c1] | A14 | 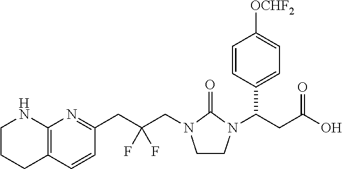 [SMILES: O=C(O)C[C@@H](c1ccc(OC(F)F)cc1)N1CCN(CC(F)(F)Cc2ccc3c(n2)NCCC3)C1=O] |

TABLE lb

| Cmpd No. | Chemical Structure | Cmpd No. | Chemical Structure |
|---|---|---|---|
| A15 | 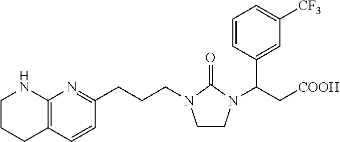 [SMILES: O=C(O)CC(c1cccc(C(F)(F)F)c1)N1CCN(CCCc2ccc3c(n2)NCCC3)C1=O] | A15s | 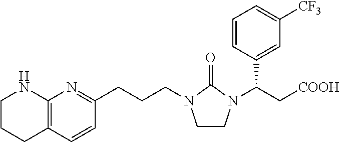 [SMILES: O=C(O)C[C@@H](c1cccc(C(F)(F)F)c1)N1CCN(CCCc2ccc3c(n2)NCCC3)C1=O] |
| A16 | 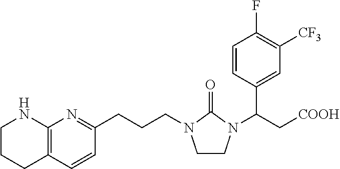 [SMILES: O=C(O)CC(c1ccc(F)c(C(F)(F)F)c1)N1CCN(CCCc2ccc3c(n2)NCCC3)C1=O] | A16s | 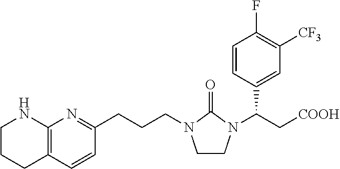 [SMILES: O=C(O)C[C@@H](c1ccc(F)c(C(F)(F)F)c1)N1CCN(CCCc2ccc3c(n2)NCCC3)C1=O] |
| A17 | 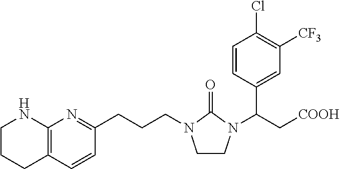 [SMILES: O=C(O)CC(c1ccc(Cl)c(C(F)(F)F)c1)N1CCN(CCCc2ccc3c(n2)NCCC3)C1=O] | A17s | 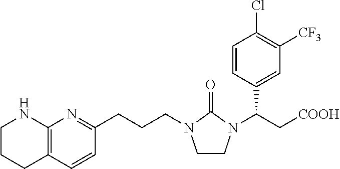 [SMILES: O=C(O)C[C@@H](c1ccc(Cl)c(C(F)(F)F)c1)N1CCN(CCCc2ccc3c(n2)NCCC3)C1=O] |
| A18 | 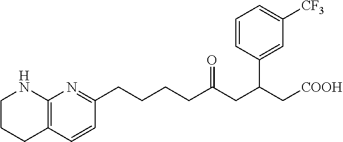 [SMILES: O=C(O)CC(CC(=O)CCCCc1ccc2c(n1)NCCC2)c1cccc(C(F)(F)F)c1] | A18s | 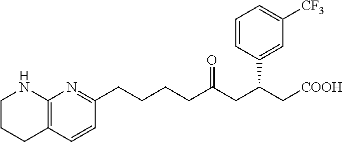 [SMILES: O=C(O)C[C@H](CC(=O)CCCCc1ccc2c(n1)NCCC2)c1cccc(C(F)(F)F)c1] |
| A19 | 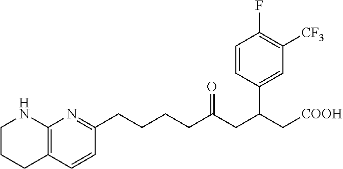 [SMILES: O=C(O)CC(CC(=O)CCCCc1ccc2c(n1)NCCC2)c1ccc(F)c(C(F)(F)F)c1] | A19s | 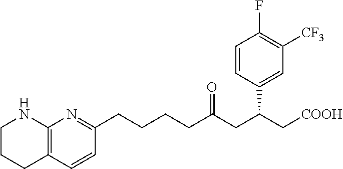 [SMILES: O=C(O)C[C@H](CC(=O)CCCCc1ccc2c(n1)NCCC2)c1ccc(F)c(C(F)(F)F)c1] |
| A20 | 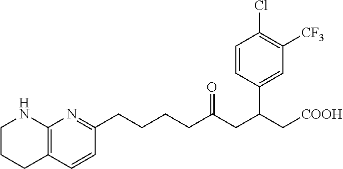 [SMILES: O=C(O)CC(CC(=O)CCCCc1ccc2c(n1)NCCC2)c1ccc(Cl)c(C(F)(F)F)c1] | A20s | 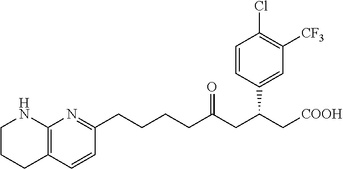 [SMILES: O=C(O)C[C@H](CC(=O)CCCCc1ccc2c(n1)NCCC2)c1ccc(Cl)c(C(F)(F)F)c1] |
| A21 | 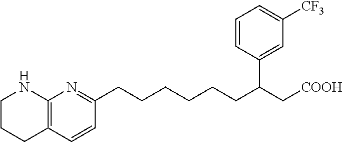 [SMILES: O=C(O)CC(CCCCCCc1ccc2c(n1)NCCC2)c1cccc(C(F)(F)F)c1] | A21s | 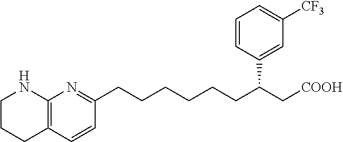 [SMILES: O=C(O)C[C@H](CCCCCCc1ccc2c(n1)NCCC2)c1cccc(C(F)(F)F)c1] |
| A21-1 enantiomer 1 | 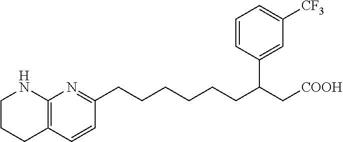 [SMILES: O=C(O)CC(CCCCCCc1ccc2c(n1)NCCC2)c1cccc(C(F)(F)F)c1] | A21-2 enantiomer 2 | 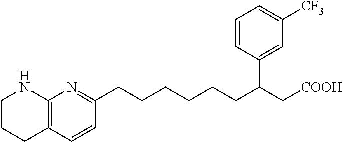 [SMILES: O=C(O)CC(CCCCCCc1ccc2c(n1)NCCC2)c1cccc(C(F)(F)F)c1] |
| A22 | 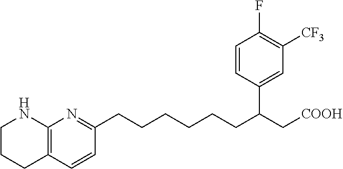 [SMILES: O=C(O)CC(CCCCCCc1ccc2c(n1)NCCC2)c1ccc(F)c(C(F)(F)F)c1] | A22s | 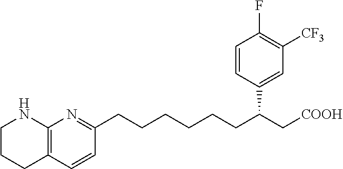 [SMILES: O=C(O)C[C@H](CCCCCCc1ccc2c(n1)NCCC2)c1ccc(F)c(C(F)(F)F)c1] |
| A23 | 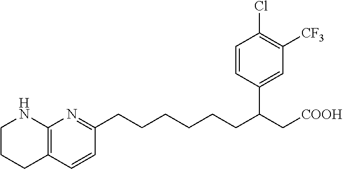 [SMILES: O=C(O)CC(CCCCCCc1ccc2c(n1)NCCC2)c1ccc(Cl)c(C(F)(F)F)c1] | A23s | 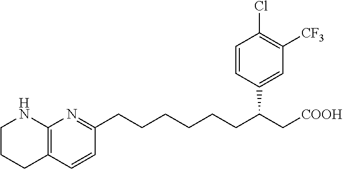 [SMILES: O=C(O)C[C@H](CCCCCCc1ccc2c(n1)NCCC2)c1ccc(Cl)c(C(F)(F)F)c1] |
| A24 | 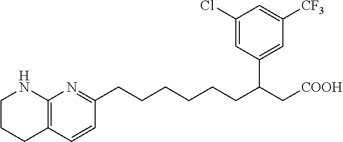 [SMILES: O=C(O)CC(CCCCCCc1ccc2c(n1)NCCC2)c1cc(Cl)cc(C(F)(F)F)c1] | A24s | 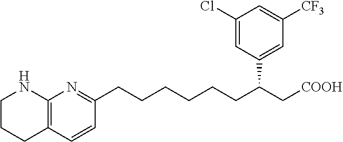 [SMILES: O=C(O)C[C@H](CCCCCCc1ccc2c(n1)NCCC2)c1cc(Cl)cc(C(F)(F)F)c1] |
| A25 | 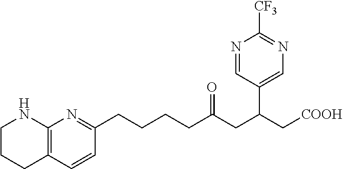 [SMILES: O=C(O)CC(CC(=O)CCCCc1ccc2c(n1)NCCC2)c1cnc(C(F)(F)F)nc1] | A25s | 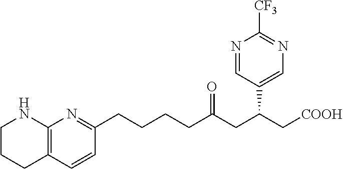 [SMILES: O=C(O)C[C@H](CC(=O)CCCCc1ccc2c(n1)NCCC2)c1cnc(C(F)(F)F)nc1] |
| A26 | 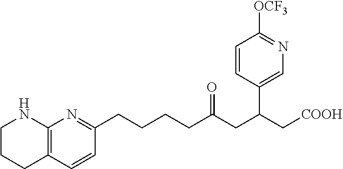 [SMILES: O=C(O)CC(CC(=O)CCCCc1ccc2c(n1)NCCC2)c1ccc(OC(F)(F)F)nc1] | A26s | 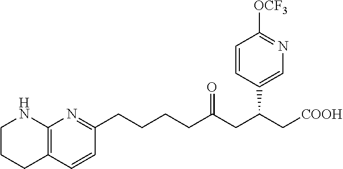 [SMILES: O=C(O)C[C@H](CC(=O)CCCCc1ccc2c(n1)NCCC2)c1ccc(OC(F)(F)F)nc1] |
| A27 | 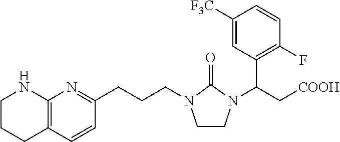 [SMILES: O=C(O)CC(c1cc(C(F)(F)F)ccc1F)N1CCN(CCCc2ccc3c(n2)NCCC3)C1=O] | A27s | 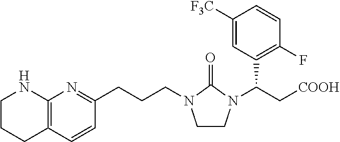 [SMILES: O=C(O)C[C@@H](c1cc(C(F)(F)F)ccc1F)N1CCN(CCCc2ccc3c(n2)NCCC3)C1=O] |
| A28 | 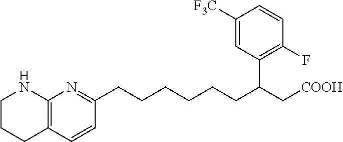 [SMILES: O=C(O)CC(CCCCCCc1ccc2c(n1)NCCC2)c1cc(C(F)(F)F)ccc1F] | A28s | 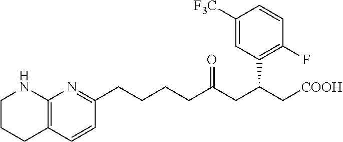 [SMILES: O=C(O)C[C@H](CC(=O)CCCCc1ccc2c(n1)NCCC2)c1cc(C(F)(F)F)ccc1F] |
| A29 | 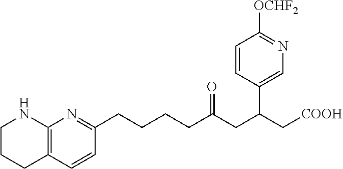 [SMILES: O=C(O)CC(CC(=O)CCCCc1ccc2c(n1)NCCC2)c1ccc(OC(F)F)nc1] | A29s | 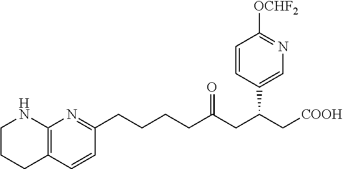 [SMILES: O=C(O)C[C@H](CC(=O)CCCCc1ccc2c(n1)NCCC2)c1ccc(OC(F)F)nc1] |
| A30 | 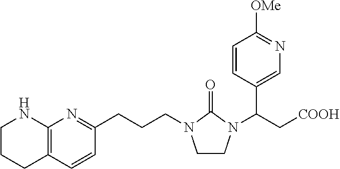 [SMILES: COc1ccc(C(CC(=O)O)N2CCN(CCCc3ccc4c(n3)NCCC4)C2=O)cn1] | A30s | 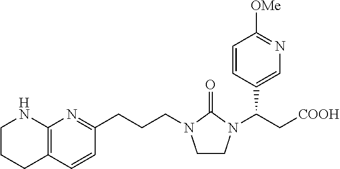 [SMILES: COc1ccc([C@H](CC(=O)O)N2CCN(CCCc3ccc4c(n3)NCCC4)C2=O)cn1] |

19. The method of claim 1, wherein the integrin inhibitor is Compound A1: 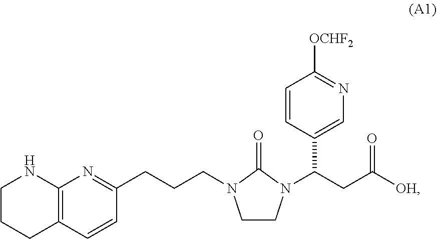 [SMILES: O=C(O)C[C@@H](c1ccc(OC(F)F)nc1)N1CCN(CCCc2ccc3c(n2)NCCC3)C1=O]
or a pharmaceutically acceptable salt thereof.

20-27. (canceled)

28. A method of treating or preventing a viral infection by SARS-CoV-2 or a condition or symptom associated with SARS-CoV-2 infection in a subject in need thereof, comprising administering to the subject a therapeutically effective amount of Compound A1: 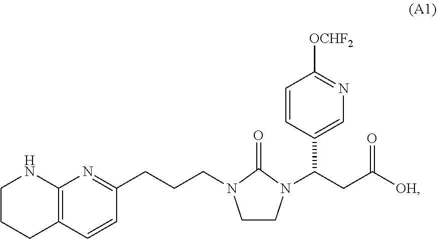 [SMILES: O=C(O)C[C@@H](c1ccc(OC(F)F)nc1)N1CCN(CCCc2ccc3c(n2)NCCC3)C1=O]
or a pharmaceutically acceptable salt or solvate thereof.

29-32. (canceled)

33. The method of claim 28, wherein the method comprises administering to the subject a therapeutically effective amount of Compound A1: 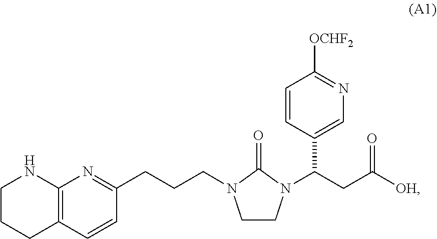 [SMILES: O=C(O)C[C@@H](c1ccc(OC(F)F)nc1)N1CCN(CCCc2ccc3c(n2)NCCC3)C1=O]
or a pharmaceutically acceptable salt thereof.
